SEQUENCE LISTING (11 sequences)

SEQ ID NO: 1 - DNA, 20 bp, synthetic construct
  source 1..20: mol_type = other DNA
cattgcccactgatgctgtg

SEQ ID NO: 2 - DNA, 21 bp, synthetic construct
  source 1..21: mol_type = other DNA
aggctttctaccccatcttga

SEQ ID NO: 3 - DNA, 23 bp, synthetic construct
  source 1..23: mol_type = other DNA
gaggcactccttaaaattctggg

SEQ ID NO: 4 - DNA, 21 bp, synthetic construct
  source 1..21: mol_type = other DNA
gcccttccacgattgtactcg

SEQ ID NO: 5 - DNA, 20 bp, synthetic construct
  source 1..20: mol_type = other DNA
gacaagcttcccgttctcag

SEQ ID NO: 6 - DNA, 20 bp, synthetic construct
  source 1..20: mol_type = other DNA
gagtcaacggatttggtcgt

SEQ ID NO: 7 - RNA, 15 nt, synthetic construct
  source 1..15: mol_type = other RNA
aaagaggacaccctg

SEQ ID NO: 8 - RNA, 15 nt, synthetic construct
  source 1..15: mol_type = other RNA
tgaatggacagctca

SEQ ID NO: 9 - RNA, 15 nt, synthetic construct
  source 1..15: mol_type = other RNA
gccacggactttcag

SEQ ID NO: 10 - RNA, 15 nt, synthetic construct
  source 1..15: mol_type = other RNA
gggctggactcatcg

SEQ ID NO: 11 - RNA, 1236 nt, Homo sapiens
  source 1..1236: mol_type = mRNA
tccgagagcgtggccacatgctggagaaccacgtcgagcgcctctgggcctacctcaccatccaggagctgctggccaagcggatgaaggtggacagggaggagagggccaacctgtcatcccaggccctgcagatgtcgctggactatgggtttgtgaccccactgacctccatgagcatcaggggcatggcggaccaggacggcctgaagcccaccatcgacaagccctcagaggattctccgcctttggagatgctgggacccagaaggacgttcgtgctgtcagccttgcagccttctcctactcattccagctccaatacccagcggctgccagaccgagtgaccggcgtggacacagaccctcacttcatcatccacgtgccccagaaagaggacaccctgtgcttcaacatcaatgaggagcctggtgttatcctgagcctggtacaggaccccaacacaggcttctcagtgaatggacagctcattggcaacaaggccaggagccctgggcagcatgacggcacgtacttcgggcggctgggaatcgcaaaccctgccacggactttcagttggaagtgactcctcagaacattacgctgaaccccggctttggtgggcctgtgttttcctggagggaccaagctgtgctgcggcaggacggggtggtggtgaccatcaacaagaagaggaacctggtggtgtctgtggacgacggtggcacctttgaggttgttttgcaccgagtgtggaaggggagctcggtccaccaggacttcctgggcttctatgtgctggacagtcatcggatgtcagcccggacgcacgggctgctggggcaatttttccaccccatcggttttgaagtgtctgacatccacccaggctctgaccccacaaagccagatgccacgatggtggtgaggaaccgccggctcacggtcaccaggggtttgcaaaaagactacagcaaggacccgtggcatggggccgaggtgtcctgctggttcattcacaacaatggggctggactcatcgatggtgcctacactgattatatcgtccccgacatcttctgagccctctggccagcacgcctgtcctcccccggggccaaggcagaggaggaggacgacatcctgacctgctgctgaggctgtacctccttgactaagctggttccttgtgtcaaagcacctcatgccttccattaaagagaggccgtgtcca

(19) United States
(12) Patent Application Publication
(10) Pub. No.: US 2026/0042844 A1
(43) Pub. Date: Feb. 12, 2026

(54) APPLICATION OF ITIH1 IN PREPARATION OF DRUG FOR TREATMENT OF HEPATOCELLULAR CARCINOMA

(71) Applicant: Tongji Hospital affiliated to Tongji Medical College of Huazhong University of Science and Technolog, Wuhan (CN)

(72) Inventors: Shuilin Dong, Wuhan (CN); Zhibin Liao, Fuzhou (CN); Furong Liu, Ji'an (CN); Zhanguo Zhang, Wuhan (CN)

(21) Appl. No.: 19/292,946

(22) Filed: Aug. 7, 2025

(30) Foreign Application Priority Data
Aug. 8, 2024 (CN) 2024110851297

(51) Int. Cl. C07K 16/28; A61K 9/00; A61K 31/7088; A61K 38/00; A61P 35/04

(52) U.S. Cl. CPC C07K 16/2842; A61K 9/0019; A61K 31/7088; A61K 38/00; A61P 35/04; C07K 2317/51

(57) ABSTRACT

An application of inter-alpha-trypsin inhibitor heavy chain 1 (ITIH1) in preparing a drug for treating hepatocellular carcinoma (HCC) is provided, relating to the field of biomedical technologies. The drug includes the ITIH1 or a reagent for increasing an expression of the ITIH1. The reagent includes a small molecule compound, an antibody drug, a protein, a nucleic acid molecule, a polypeptide, a lipid, a carbohydrate or a combination thereof. It proves a new function of ITIH1, that is, ITIH1 can act as a tumor suppressor in the progression of HCC. Moreover, in preclinical models (mouse model, human-like organ and human-like tissue xenotransplantation), it is confirmed that the purified recombinant ITIH1 (r-ITIH1) protein can be used to treat HCC. Based on the function of ITIH1, it can provide a target for developing drugs to alleviate and/or treat HCC.

### CROSS-REFERENCE TO RELATED APPLICATION

This application claims priority to Chinese Patent Application No. 202411085129.7, filed Aug. 8, 2024, which is herein incorporated by reference in its entirety.

### TECHNICAL FIELD

The disclosure relates to the field of biomedical technologies, and more particularly to an application of inter-alpha-trypsin inhibitor heavy chain 1 (ITIH1) in preparation of a drug for treatment of hepatocellular carcinoma.

### STATEMENT REGARDING SEQUENCE LISTING

The sequence listing associated with this application is provided in text format in lieu of a paper copy and is hereby incorporated by reference into the specification. The name of the XML file containing the sequence listing is 25051THXT-USP1-SL.xml. The XML file is 11,988 bytes; is created on Aug. 6, 2025; and is being submitted electronically via patent center.

### BACKGROUND

Hepatocellular carcinoma (HCC) is one of the most common malignant tumors worldwide. It ranks as the sixth most frequently diagnosed cancer and the fourth leading cause of cancer-related deaths. Although great efforts have been made in the past decades to develop therapeutic strategies, including surgical resection, liver transplantation, new therapeutic drugs and comprehensive treatment, the five-year survival rate remains unsatisfactory.

Therefore, elucidating the molecular mechanisms underlying HCC and identifying novel therapeutic options are crucial for improving patient outcomes.

### SUMMARY

In view of this, the main objective of the disclosure is to provide an application of ITIH1 in preparing a drug for treating hepatocellular carcinoma (HCC). By determining the regulatory effect of the expression of ITIH1 on HCC, the ITIH1 gene or protein is used for the prevention and treatment of HCC, specifically the application of ITIH1 as a target in the preparation of drugs for preventing, relieving or treating HCC.

In order to achieve the above objective, the disclosure provides an application of ITIH1 in preparing drugs for treating HCC.

In an embodiment, the drug includes the ITIH1 or a reagent for increasing an expression of the ITIH1.

In an embodiment, the reagent includes a small molecule compound, an antibody drug, a protein, a nucleic acid molecule, a polypeptide, a lipid, a carbohydrate or a combination thereof.

In an embodiment, the ITIH1 is used to influence the metastasis of HCC by competitively binding to integrin subunit beta 1 (ITGB1)/integrin subunit alpha 5 (ITGA5) complex and inhibiting integrin/focal adhesion kinase (FAK) signaling pathway.

In an embodiment, an expression of the ITIH1 is regulated by transforming growth factor-beta (TGF-β) stimulation and N6-methyladenosine (m6A) modification.

The disclosure also provides an application of ITIH1 as a target in developing and screening a drug for treating HCC.

The disclosure also provides an application of one of an ITIH1 protein, an ITIH1 gene, and a biomaterial related the ITIH1 gene in preparing a drug for treating HCC.

The disclosure also provides an application of a substance taking one of an ITIH1 protein and an ITIH1 gene as a target in preparing a drug. The target is an up-regulation target or an activation target. The drug is indicated for treating HCC.

During application, the reagent is galunisertib, which is purchased from Selleck and administered at a dose of 50 milligrams per kilogram (mg/kg). It is dissolved in dimethyl sulfoxide (DMSO) as a stock solution and diluted with corn oil to the use concentration. It is injected intraperitoneally into mice three times a week.

ITIH1 is synthesized and administered at a dose of 20 micrograms (μg)/mouse. The purified protein is dissolved in phosphate buffered saline (PBS) three times a week and injected into the tail vein of mice.

The disclosure also provides a drug, which includes a substance taking one of an ITIH1 protein and an ITIH1 gene as a target, the target is an up-regulation target or an activation target, and the drug is indicated for treating HCC.

The disclosure also provides a drug, which includes one of an ITIH1 protein, an ITIH1 gene, and a biomaterial related the ITIH1 gene. The drug is indicated for treating HCC.

Beneficial effects of that disclosure at least are as follows.

The disclosure finds a new function of ITIH1, that is, ITIH1 can act as a tumor suppressor in the progression of HCC. In preclinical models (mouse model, human-like organ and human-like tissue xenotransplantation), it is confirmed that the purified recombinant ITIH1 (r-ITIH1) protein can be used to treat HCC.

Based on the function of ITIH1, it can provide a target for developing drugs to alleviate and/or treat HCC.

### BRIEF DESCRIPTION OF DRAWINGS

FIGS. 1A-1L illustrate results of down-regulation of ITIH1 by TGF-β stimulation according to an embodiment of the disclosure. Specifically, FIG. 1A illustrates co-immunoprecipitation (co-IP) results of methyltransferase like 3 (METTL3) and SMAD family member 2/3 (SMAD2/3). FIG. 1B illustrates co-IP results of Flag-SMAD3 and HA-METTL3 in 293T cells. FIG. 1C illustrates co-IP results of Flag-SMAD3 and HA-METTL3 in 293T cells with or without TGF-β stimulation for 12 hours (h). FIG. 1D illustrates co-IP results of Flag-SMAD3 and HA-METTL3 in 293T cells with or without TGF-β stimulation for 12 h and galunisertib (also referred to as LY2157299) treatment for 12 h. FIG. 1E illustrates endogenous co-IP results of SMAD3 and METTL3 with or without TGF-β stimulation for 12 h. FIG. 1F illustrates representative images of METTL3-enhanced yellow fluorescent protein (METTL3-EYFP) aggregates in cells transfected with small interfering RNA targeting SMAD3 (si-SMAD3) or treated with TGF-β for 12 h. FIG. 1G illustrates intersection of two RNA-seq data sets, 97H-Con vs. 97H-shSMAD3 and 97H-Con vs. 97H-shMETTL3. FIG. 1H illustrates messenger RNA (mRNA) expression of ITIH1 in human liver fibroblasts (HLFs) and 97H cells changed in a time-dependent manner under TGF-β stimulation. FIG. 1I illustrates relative luciferase activity of HLF cells transfected with pGL4.17 plasmid containing ITIH1 promoter region after TGF-β stimulation for 12 h. FIG. 1J illustrates relative changes of mRNA expression of ITIH1 in HLFs when SMAD2 or SMAD3 is knocked out or overexpressed. FIG. 1K illustrates relative changes of mRNA expression of ITIH1 in HLFs and 97H cells transfected with plasmids. FIG. 1L illustrates representative results of immunohistochemistry (IHC) staining of ITIH1 in paraffin-embedded HCC samples from Tongji Hospital affiliated to Tongji Medical College of Huazhong University of Science & Technology, in which the right portion in FIG. 1L shows statistical results of expression in non-tumor tissue (N) and tumor tissue (T), and the following charts show the correlation between ITIH1 and METTL3/SMAD3 expression determined from IHC results. Data are presented as mean±standard deviation (SD) in bar graphs. ns: not significant; *: P<0.05; **: P<0.01; ***: P<0.001. M3: METTL3; Gal: galunisertib.

FIGS. 2A-2F illustrate correlation among METTL3, SMAD3 and ITIH1 according to the embodiment of the disclosure. Specifically, FIG. 2A illustrates phosphorylated (activated) and total SMAD2/SMAD3 levels in HLFs and 97H cells after transfection of METTL3 and AlkB Homolog 5, RNA demethylase (ALKBH5) and TGF-β stimulation for 12 h by Western Blot (WB). FIG. 2B illustrates relative mRNA expression levels of genes in 97H-Con and 97H-shSMAD3 cells in FIG. 1G. FIG. 2C illustrates relative mRNA expression levels of genes in 97H-Con and 97H-shMETTL3 cells in FIG. 1G. FIG. 2D illustrates the correlation between predicted ITIH1 and METTL3 expression and the correlation between ITIH1 and SMAD3 expression by gene expression profiling interactive analysis (GEPIA). FIGS. 2E-2F illustrate representative results of IHC staining of SMAD3/METTL3 in paraffin-embedded HCC samples in Tongji Hospital affiliated to Tongji Medical College of Huazhong University of Science & Technology, in which statistical results of expression in non-tumor tissue (N) and tumor tissue (T) are shown in the right portions of FIGS. 2E-2F.

FIGS. 3A-31 illustrate results of METTL3 regulating ITIH1 expression of in a m6A-dependent manner in the embodiment of the disclosure. Specifically, FIG. 3A illustrates relative mRNA expression level of ITIH1 when METTL3 is knocked out or overexpressed. FIG. 3B illustrates relative expression level of ITIH1 after cells are transfected with METTL3-wt (wild type) and METTL3-mut (catalytic site mutation) plasmids respectively. FIG. 3C illustrates relative luciferase activity stimulated by TGF-β or galunisertib after the cells are co-transfected with psiCHECK2 and METTL3 plasmids containing ITIH1 mRNA sequence. FIG. 3D illustrates relative mRNA expression level of ITIH1 stimulated by TGF-β or galunisertib (the left two portions) and the level of ITIH1 protein (the rightmost portion) after 293T cells are transfected with different concentrations of METTL3-wt or METTL3-mut. FIG. 3E illustrates a schematic diagram of truncated mutants of ITIH1. FIG. 3F illustrates protein levels of cells co-transfected with HA-METTL3 and Flag-ITIH1-full length (FL)/M5 in a concentration-dependent manner. FIG. 3G illustrates relative luciferase activity of cells transfected with psiCHECK2 containing ITIH1-wt or ITIH1-M5 and overexpressing METTL3-WT. FIG. 3H illustrates results of RNA pulldown using ITIH1-wt or ITIH1-M5. FIG. 3I illustrates methylated RNA immunoprecipitation (MeRIP) results in cells co-transfected with ITIH1-M5 and METTL3-Mut. Data are presented as mean±standard deviation (SD) in bar graphs. ns: not significant; *: P<0.05; **: P<0.01; ***: P<0.001. Gal: galunisertib.

FIGS. 4A-4B illustrate predicted m6A sites in ITIH1. Specifically, FIG. 4A illustrates m6A sites in ITIH1 mRNA predicted by a sequence-based RNA modification site predictor (SRAMP). FIG. 4B illustrates possible m6A sites in ITIH1 mRNA that are pointed out.

FIGS. 5A-5B illustrate WB verification of m6A sites in ITIH1. Specifically, FIG. 5A illustrates protein levels of cells co-transfected with HA-METTL3 and Flag-ITIH1-M1/M2/M3/M4 in a concentration-dependent manner. FIG. 5B illustrates protein levels of cells co-transfected with HA-METTL3 and ITIH1 truncation mutant plasmids in a concentration-dependent manner.

FIGS. 6A-6H illustrate degradation results of ITIH1 mRNA promoted by YTH domain family member 1 (YTHDF2) in an M6A-dependent manner according to the embodiment of the disclosure. Specifically, FIG. 6A illustrates remaining ITIH1 mRNA level in 97H-shMETTL3 cells after treatment with TGF-β, galunisertib and actinomycin D for a specified time. FIG. 6B illustrates remaining ITIH1 mRNA level in 97H-oeMETTL3 cells after treatment with TGF-β, galunisertib and actinomycin D for a specified time. FIG. 6C illustrates relative mRNA expression level of ITIH1 in 97H cells treated with YTHDF1 or YTHDF2 siRNA. FIG. 6D illustrates results of RNA pulldown using ITIH1-wt or ITIH1-M5. FIG. 6E illustrates relative changes of ITIH1 level in 97H-shMETTL3 cells treated with TGF-β or galunisertib determined by RNA immunoprecipitation (RIP) using anti-YTHDF2 antibody. FIG. 6F illustrates a schematic diagram of YTHDF2-wt (wild type) and YTHDF2-mut (mutant type). FIG. 6G illustrates relative mRNA levels of YTHDF2 and ITIH1 after 97H cells are co-transfected with YTHDF2-mut and ITIH1-M5. FIG. 6H illustrates protein levels of YTHDF2 and ITIH1 after 97H cells are co-transfected with YTHDF2-mut and ITIH1-M5. Data are presented as mean±standard deviation (SD) in bar graphs. ns: not significant; *: P<0.05; **: P<0.01; ***: P<0.001. Gal: galunisertib.

FIGS. 7A-7D illustrate correlation between YTHDF2/METTL3 expression and YTHDF2/ITIH1 expression. Specifically, FIG. 7A illustrates correlation between METTL3 and YTHDF2 expression from GEPIA. FIG. 7B illustrates correlation between ITIH1 and YTHDF2 expression from GEPIA. FIG. 7C illustrates disease-free survival (DFS) and overall survival (OS) of patients with low or high expression level of METTL3 from GEPIA. FIG. 7D illustrates DFS and OS of patients with low or high expression level of YTHDF2 from GEPIA. DFS usually refers to the disease-free survival, which refers to the time from the end of treatment to the recurrence of the disease. OS refers to the overall survival, that is, the time from diagnosis or treatment to death. GEPIA is an online database used to analyze the correlation between gene expression level and clinical results.

FIGS. 8A-8J illustrate results of ITIH1 inhibiting HCC progression both in vitro and in vivo. Specifically, FIG. 8A illustrates statistical results of transwell migration and invasion experiments in Hep3B-shITIH1, HLF-oeITIH1, and 97H-oeITIH1 cells. FIG. 8B illustrates statistical results of wound healing experiments in specified stable cell lines. FIG. 8C illustrates results of orthotopic transplantation model and tail vein injection model established in nude mice using 97H-luc-ITIH1 or vector cells, in which charts below show statistical results, the upper portions show representative images of lung metastasis by hematoxylin and eosin (H&E) staining. FIG. 8D illustrates collection of livers from hydrodynamic tail vein injection (HTVi) model for weighing and IHC staining. FIG. 8E illustrates proportions of programmed cell death protein 1-positive (PD-1⁺) cells in cluster of differentiation 8-positive (CD8⁺) T cells detected by flow cytometry. FIG. 8F illustrates representative images of HCC-like organs treated with PBS or r-ITIH1 for 10 days, and quantification of the diameter of the HCC-like organs. FIG. 8G illustrates a schematic diagram of generation of a patient-derived xenograft (PDX) model. Patient-derived tumor tissues are xenografted and passaged in NOD-Prkdcem26/NjuCrl (NCG) mice, a strain of severely immunodeficient mice. FIG. 8H illustrates the PDX model administered with or without r-ITIH1 in NCG mice, after 25 days of intervention, tumor volume and weight in the liver are measured. FIG. 8I illustrates calculated tumor volume and tumor weight after each administration. FIG. 8J illustrates results of H&E and hepatocyte staining performed in the tumor. Data are presented as mean±standard deviation (SD) in bar graphs. ns: not significant; *: P<0.05; **: P<0.01; ***: P<0.001.

FIGS. 9A-9E illustrate effects of ITIH1 in vitro and in vivo. Specifically, FIG. 9A illustrates protein levels of ITIH1 in HCC cell lines detected by WB. FIG. 9B illustrates verification of levels of ITIH1 in Hep3B-shITIH1, HLF-oeITIH1 and 97H-oeITIH1 cells. FIGS. 9C-9D illustrate results of wound healing and transwell experiments in specified cell lines.

FIGS. 10A-10C illustrate influence of ITIH1 on the immune microenvironment of HCC and purification of r-ITIH1. Specifically, FIG. 10A illustrates proportions of various immune cell subsets in tumors of control group and ITIH1 overexpression group measured by flow cytometry. FIG. 10B illustrates proportion of CD8⁺ T cells in tumors of control group and ITIH1 overexpression group was detected by flow cytometry. FIG. 10C illustrates Coomassie brilliant blue staining of r-ITIH1.

FIGS. 11A-11F illustrate results of interaction between ITIH1 and integrin α5β1. Specifically, FIG. 11A illustrates silver staining results after IP. FIG. 11B illustrates results of IP with HA-ITIH1+Flag-ITGA5 and HA-ITIH1+Flag-ITGB1 in 293T cells. FIG. 11B illustrates immunofluorescence (IF) stained confocal images of ITIH1+ITGB1 and ITIH1+ITGA5 in HLF cells. FIG. 11D illustrates a schematic diagram of construction of truncated mutants of ITGA5 and ITGB1, IP results are obtained by constructing truncated mutants with ITIH1 and ITGB1 and ITGA5. FIG. 11E illustrates IP results of ITIH1+ITGB1 and ITGB1+FN1 when fibronectin 1 (FN1) or r-ITIH1 is added in a concentration gradient. FIG. 11F illustrates results of IP in 293T cells using ITGB1-wt or deletion mutant of ITGB1 and FN1 or ITIH1. Data are presented as mean±standard deviation (SD) in bar graphs. ns: not significant; *: P<0.05; **: P<0.01; ***: P<0.001.

FIGS. 12A-12D illustrate verification of the interaction between ITIH1 and integrin family members. Specifically, FIG. 12A illustrates results of IP with HA-ITIH1 and other specified proteins in 293T cells. FIG. 12B illustrates results of IP with ITIH1, ITGB1 and ITGA5 in HLF cells. FIG. 12C illustrates IP results in 293T cells using ITIH1 and ITGB3. FIG. 12D illustrates IP results of ITIH1+ITGA5 and ITGA5+FN1 when FN1 or r-ITIH1 is added in a concentration gradient.

FIGS. 13A-13G illustrate related experimental results according to the embodiment of the disclosure. Specifically, FIG. 13A illustrates representative IHC staining results of paraffin-embedded HCC samples in Tongji Hospital affiliated to Tongji Medical College of Huazhong University of Science & Technology. FIG. 13B illustrates correlation between pFAK/ITIH1 and pSRC/ITIH1 levels determined by IHC staining. FIG. 13C illustrates a Waterfall diagram showing clinicopathological features significantly related to the expression of ITIH1 in HCC samples from Tongji Hospital affiliated to Tongji Medical College of Huazhong University of Science & Technology. FIG. 13D illustrates multivariate regression analysis, which analyzes the clinicopathological features determined by univariate regression analysis that are significantly related to the prognosis of HCC samples in Tongji Hospital affiliated to Tongji Medical College of Huazhong University of Science & Technology. FIG. 13E illustrates OS prognosis of patients according to the protein levels of ITIH1, pFAK and pSRC. FIG. 13F illustrates DFS prognosis of patients according to the protein levels of ITIH1, pFAK and pSRC. FIG. 13G illustrates a pattern diagram showing the role of ITIH1 in HCC cells. Data are presented as mean±standard deviation (SD) in bar graphs. ns: not significant; *: P<0.05; P<0.01; ***: P<0.001.

FIGS. 14A-14F illustrate correlation between ITIH1 and pFAK/pSRC protein level. Specifically, FIG. 14A illustrates patients with HCC in Tongji Hospital affiliated to Tongji Medical College of Huazhong University of Science & Technology analyzed by WB, and relative expression of ITIH1 is analyzed. FIGS. 14B-14D illustrate relative protein levels of ITIH1, pFAK and pSRC analyzed in non-tumor tissues and tumor tissues. FIGS. 14E-14F illustrate statistical correlation between PFAK/ITIH1 and pSRC/ITIH1 levels.

### DETAILED DESCRIPTION OF EMBODIMENTS

Technical solutions in embodiments of the disclosure will be described clearly and completely below. Apparently, the described embodiments are only a part of the embodiments of the disclosure, but not the whole embodiments. Based on the embodiments in the disclosure, all other embodiments obtained by those skilled in the art without creative work belong to the scope of protection of the disclosure.

In addition, the technical solutions of various embodiments of the disclosure can be combined with each other, but they must be based on the realization of those skilled in the art. When the combination of technical solutions is contradictory or impossible, it should be considered that the combination of technical solutions does not exist and is not within the scope of protection required by the disclosure.

In the following, the technical solutions proposed by the disclosure will be explained in detail through specific embodiments.

### 1. Experimental Method

### 1.1 Western Blot

### 1.1.1 Cell Protein Extraction

1). Cells to be collected for protein extraction are removed from the cell culture incubator, washed twice with pre-cooled PBS, and the PBS is carefully aspirated. Radioimmunoprecipitation assay (RIPA) lysis buffer is then added, and the cells are lysed on ice for 30 minutes (min).
2). The cells are scraped from the culture dish using a cell scraper and centrifuged at 12,000 revolutions per minute (rpm) at 4° C. for 15 min.
3). The supernatant is collected for subsequent experiments and can also be stored at −80° C.

### 1.1.2 Protein Quantification

Protein quantification is performed using the bicinchoninic acid assay (BCA) method with a kit purchased from Thermo Fisher (Cat. No. 23225).
1) Protein standard solutions are prepared by diluting the protein standard (2 milligrams per milliliter, abbreviated as mg/mL) provided in the kit with distilled water to achieve the following concentration gradients: 1000 nanograms per microliter abbreviated as ng/μL, 750 ng/μL, 500 ng/μL, 250 ng/μL, 125 ng/μL, 62.5 ng/μL, 31.25 ng/μL, and 0 ng/μL. These standards are stored at 4° C.
2) The protein samples to be tested are diluted 10-fold with double-distilled water (12 microliters abbreviated as μL of protein sample mixed with 108 μL of double-distilled water).
3) A volume of 50 μL of each gradient protein standard and diluted protein sample is added to the wells.
4) Reagent A and Reagent B from the BCA kit are mixed thoroughly at a ratio of 50:1.

Then, 200 μL of the mixture is added to each well of a 96-well plate, followed by incubation at 37° C. for 30 min.
5) The absorbance values of the proteins are measured.
6) The concentrations of the protein samples are calculated using formulas in Excel.
7) The protein concentrations are adjusted to 2 micrograms per microliter abbreviated as μg/μL. The required volume of the original protein solution is calculated and transferred. 4× loading buffer and RIPA lysis buffer are added to reach the final volume, and the mixture is vortexed thoroughly. The samples are boiled for 10 min, cooled on ice, and then used for subsequent experiments or stored at −80° C.

### 1.1.3 Gel Preparation, Electrophoresis and Film Transfer

### Gel Preparation

a. A sodium dodecyl sulfate-polyacrylamide gel electrophoresis (SDS-PAGE) gel preparation kit is purchased from Wuhan Boster Biological Technology Co., Ltd. (Cat. No. AR0138) and used to prepare the stacking gel and resolving gel (also referred to as separating gel) according to the manufacturer's instructions.
b. Electrophoresis Buffer (1000 mL system):

| | | |
|---|---|---|
| Double-distilled water | 1000 | mL |
| Glycine | 18.87 | grams (g) |
| Tris-base | 3.03 | g |
| SDS | 1 | g |

Transfer Buffer (1000 mL system):

| | | |
|---|---|---|
| Glycine | 14.42 | g |
| Tris-base | 2.9 | g |
| Double-distilled water | 800 | mL |
| Methanol (added during transfer) | 200 | mL |

### Sample Loading and Electrophoresis

1) The electrophoresis tank is filled with an appropriate amount of electrophoresis buffer, and the comb is carefully removed from the stacking gel.
2) After determining the sample loading order, samples are loaded into the wells using a 10 μL pipette. Equal loading volumes are ensured for all wells, and a lane for protein marker is designated.
3) Power is connected, and electrophoresis is initiated at a constant voltage of 80 volts (V). When the protein marker bands begin to separate, the voltage is adjusted to 100 V. Electrophoresis is terminated when proteins are fully separated or have reached the bottom of the resolving gel (indicated by bromophenol blue).

### Protein Transfer

1) A piece of polyvinylidene fluoride (PVDF) membrane of appropriate size is cut and soaked in methanol for 3-5 min to activate it.
2). The power is turned off, the electrophoresis buffer is discarded, the gel plates are rinsed with tap water, and the gel is removed and placed into transfer buffer for later use.
3) The transfer cassette is opened with the black cathode side facing down, and sponges and filter papers are placed as padding. The gel is gently placed onto the filter paper. The activated PVDF membrane is briefly washed in transfer buffer, then carefully placed onto the gel. Additional filter papers and sponges are added on top. Care is taken to ensure no air bubbles are trapped between the layers. The cassette is closed and inserted into the transfer tank, which is then filled with an appropriate amount of transfer buffer.
4) Ice packs pre-frozen at −80° C. are placed into the transfer tank. Power is turned on, and transfer is performed at a constant current of 200 milliamperes (mA) for 2 hours.

### 1.1.4 Antibody Incubation and Result Detection

1) Blocking: The PVDF membrane is removed and blocked with skim milk at room temperature for 1 hour on a shaker.
2) Primary antibody incubation: The blocked PVDF membrane is washed once with Tris-Buffered Saline with Tween® 20 (TBST) and then incubated with the prepared primary antibody dilution solution overnight at 4° C.
3) Secondary antibody incubation: The PVDF membrane, after overnight incubation with the primary antibody, is washed three times with TBST, each for 10 min. It is then incubated with the secondary antibody dilution solution at 37° C. for 1 hour.
4). Results detection: The PVDF membrane, after overnight incubation with the secondary antibody, is washed three times with TBST, each for 10 min. An appropriate amount of developing solution is prepared for color development, and images are captured using a laboratory imaging system.

### 1.2 Co-Immunoprecipitation (Co-IP)

1) 293T cells or target cells transfected with plasmids are cultured for 24-48 hours. The culture medium is discarded, and the cells are washed twice with cold PBS, with excess PBS carefully removed.
2) 0.5 mL of IP lysis buffer containing protease inhibitors (and phosphatase inhibitors if required) is added, mixed gently, and the cells are lysed on ice for 30 min.
3) The cells are scraped off completely using a cell scraper and transferred into a clean 1.5 mL Eppendorf (EP) tube. The lysate is sonicated (with care to avoid excessive power), followed by centrifugation at 12,000 rpm at 4° C. for 15 min.
4) The supernatant is transferred to another EP tube to obtain the protein lysate. 30 μL of the protein lysate is mixed with an equal volume of 2× loading buffer as input and stored at −80° C.
5) 30 μL of protein A/G beads are washed twice with 1 mL of IP lysis buffer by centrifugation at 3,000 rpm at 4° C. for 5 min. The supernatant is discarded, and the beads are resuspended in the protein lysate. The mixture is incubated on a rotating shaker at 4° C. for 2 hours to block non-specific binding.
6) After centrifugation at 3,000 rpm at 4° C. for 5 min, the protein lysate is transferred to a new 1.5 mL EP tube, and the specific primary antibody is added. The mixture is incubated on a rotating shaker at 4° C. for 8 hours or overnight.
7) Another 30 μL of protein A/G beads are washed twice with 1 mL of IP lysis buffer by centrifugation at 3,000 rpm at 4° C. for 5 min. The supernatant is discarded, and the beads are added to the protein lysate that has been incubated with the primary antibody. The mixture is incubated on a rotating shaker at 4° C. for 2 hours.
8) After centrifugation at 3,000 rpm at 4° C. for 5 min, the liquid is gently aspirated. The beads are washed five times with IP wash buffer, with each wash involving 10 min of incubation on a rotating shaker at 4° C. and centrifugation at 3,000 rpm at 4° C. for 5 min. The supernatant is completely removed, and the beads are collected for downstream analysis.

### 1.3 Real-Time Polymerase Chain Reaction (PCR)

### 1.3.1 Total RNA Extraction

1) Cells designated for RNA collection are removed from the cell culture incubator. The culture medium is aspirated, and the cells are washed twice with pre-cooled PBS. Excess PBS is carefully removed. 1 mL of Trizol™ reagent is added, and the cells are lysed on ice for 10 min. The cells are then dislodged from the culture dish by pipetting and the lysate is transferred to a 1.5 mL EP tube for immediate use or stored at −80° C.
2) 200 μL of chloroform is added, the mixture is vortexed for 15 seconds(s), and then incubated at room temperature for 5 min.
3) The sample is centrifuged at 12,000 rpm at 4° C. for 15 min.
4) The upper aqueous phase is carefully transferred to a new and clean EP tube. 500 μL of isopropanol is added, mixed thoroughly by pipetting, and incubated at room temperature for 10 min. The sample is then centrifuged at 12,000 rpm for 10 min, resulting in a white RNA pellet at the bottom.
5) The supernatant is carefully aspirated, and the pellet is washed with 1 mL of 75% ethanol. The sample is vortexed to resuspend the pellet and centrifuged at 12,000 rpm at 4° C. for 5 min.
6) The supernatant is carefully aspirated, and the RNA pellet is air-dried in a biosafety cabinet for 10 min. The RNA is then dissolved in 100 μL of nuclease-free water.
7) The RNA concentration is measured, and the sample is used for subsequent experiments.

### 1.3.2 RNA Reverse Transcription

Reverse transcription is performed using a kit purchased from Nanjing Novozan Biotechnology Co., Ltd. The procedure is as follows.

RNA template denaturation (8 μL system):

| | | |
|---|---|---|
| Total RNA | 1 | μg |
| Random hexamers | 1 | μL |
| Nuclease-free water | to 8 | μL |

Reaction procedure: The mixture is heated at 65° C. for 5 min and then immediately placed on ice for rapid cooling, followed by incubation on ice for 3 min.

First-strand cDNA Synthesis (20 μL system):

| | | |
|---|---|---|
| The denatured mixture from | 8 | μL |
| the previous step | | |
| 2× RT Mix | 10 | μL |
| HiScript II Enzyme Mix | 2 | μL |

Reaction procedure: The reaction is carried out at 42° C. for 1 hour, followed by 80° C. for 5 min, and then held at 16° C. until completion.

1.3.3. Real-Time Fluorescence Quantitative PCR (qPCR)

Real-time fluorescence quantification is performed using a kit purchased from Nanjing Novozan Biotechnology Co., Ltd. The procedure is as follows.

1) Primer design
METTL3-Forword:
(SEQ ID NO: 1)
CATTGCCCACTGATGCTGTG
METTL3-Reverse:
(SEQ ID NO: 2)
AGGCTTTCTACCCCATCTTGA
ITIH1-Forword:
(SEQ ID NO: 3)
GAGGCACTCCTTAAAATTCTGGG
ITIH1-Reverse:
(SEQ ID NO: 4)
GCCCTTCCACGATTGTACTCG
GAPDH-Forword:
(SEQ ID NO: 5)
GACAAGCTTCCCGTTCTCAG
GAPDH-Reverse:
(SEQ ID NO: 6)
GAGTCAACGGATTTGGTCGT

2) qPCR reaction (10 μL system)

| | | |
|---|---|---|
| qPCR Master Mix | 5 | μL |
| Forward primer (10 micromoles | 0.3 | μL |
| per liter abbreviated as μM) | | |
| Reverse primer (10 μM) | 0.3 | μL |
| CDNA | 1 | μL |
| Nuclease-free water | 3.4 | μL |

Three technical replicates are set up according to the above reaction system. The PCR reaction procedures are as follows:
Pre-denaturation: 95° C. for 15 min.
Cycling reaction: 40 cycles of 95° C. for 15 s, 60° C. for 10 s, and 72° C. for 20 s.
Melting curve analysis: 65-95° C., increasing by 0.5° C. every 5 s.
3) Result analysis

Results are analyzed using the Bio-Rad PCR instrument and associated software.

### 1.4 Immunohistochemical (IHC) Staining

### 1.4.1. IHC Staining Procedures

1) Tissue sections are incubated at 60° C. for 20 min.
2) The tissue sections are quickly immersed in xylene for 3 min, then transferred to fresh xylene for another 3 min. They are subsequently placed in absolute ethanol for 3 minutes twice, 99% ethanol for 3 min, 75% ethanol for 3 min, and 50% ethanol for 3 min.
3) The tissue sections are transferred to a small glass jar and washed three times with Tris-buffered saline (TBS) on a shaker, 3 min per wash, in preparation for antigen retrieval.
4) The tissue sections are placed into a retrieval cassette, and ethylenediaminetetraacetic acid (EDTA) antigen retrieval solution (pH selected according to the antibody manufacturer's instructions) is added. The cassette is placed in a microwave and heated to boiling for 20 min.
5) The retrieval cassette is removed and allowed to cool to room temperature. The tissue sections are then transferred to a small glass jar and washed three times with TBS, 3 min per wash, in preparation for blocking endogenous peroxidase.
6) The tissue sections are immersed in a 3% hydrogen peroxide solution prepared in methanol, incubated in the dark for 10 min, and washed twice with TBS. A hydrophobic barrier is then drawn around the tissue samples using a histology pen.
7) The tissue sections are blocked with serum from the species in which the secondary antibody was raised or with 5% bovine serum albumin (BSA), which is gently applied to the tissue and incubated at 37° C. for 1 hour.
8) Primary antibody incubation: After the blocking solution is removed, the primary antibody prepared according to the manufacturer's instructions is gently applied to the tissue sections. The sections are placed in a humidified chamber and incubated at 4° C. overnight.
9) The tissue sections are removed and placed into a small glass jar, washed three times with TBS, 3 min per wash, and then the universal secondary antibody (anti-mouse/rabbit) is applied and incubated at room temperature for 1 hour.
10) The tissue sections are removed and placed into a small glass jar, washed three times with TBS, 3 min per wash, in preparation for color development.
11) DAB chromogen development: The 3,3′-Diaminobenzidine (DAB) solution (reagent A and B mixed in proportion, prepared fresh) is applied to the sections. Color development is performed for 1-5 min, with close monitoring and timely termination.
12) Counterstaining with hematoxylin is performed for 3 min. The sections are rinsed in tap water for 10 min, dipped in 1% hydrochloric acid alcohol for 5-10 s, rinsed again with tap water, placed in dilute ammonia water for 5-10 s, and finally rinsed with tap water.
13) The sections are dehydrated in a graded series: 50% ethanol for 3 min, 75% ethanol for 3 min, 95% ethanol for 3 min, absolute ethanol for 3 min twice, and xylene for 3 min twice.
14) The tissue sections are mounted with a coverslip, and images are captured under a microscope.

### 1.4.2 IHC Scoring of Tissue Microarrays (TMAs)

The immunohistochemistry score for tissue sections is calculated as the product of the staining intensity score and the percentage score of positive stained cells.

Staining intensity score: 0 for negative, 1 for weak, 2 for moderate, and 3 for strong.

Percentage of positive cells score: 0 for less than 5%, 1 for 5%-25%, 2 for 26%-50%, 3 for 51%-75%, and 4 for greater than 75%.

Scoring is independently performed by two individuals. Grouping into high-expression and low-expression groups is determined based on a consensus of the two scorers' results. Typically, a total score ≥6 is classified as high expression, while a total score <6 is classified as low expression.

### 1.5 Cell Transfection and Selection of Stable Cell Lines

1) Inoculation of target cells

The target cells are thawed and cultured until they reach good confluence and growth condition. The cells are then trypsinized, counted, and seeded into 6-well plates at a density suitable for reaching approximately 50% confluence by the next day.
2) The original culture medium in the 6-well plate is aspirated, and an appropriate volume of lentiviral supernatant is added. The plate is gently mixed and incubated for 24 hours.
3) After 24 hours of infection, the viral medium is carefully aspirated (and properly disposed of), replaced with fresh culture medium, and the cells are cultured for an additional 24 hours.
4) The original medium is aspirated and replaced with fresh culture medium containing puromycin (5 μg/mL) for selection. Cell growth is monitored daily, and the medium supplemented with puromycin is replaced regularly. After 1-2 weeks of continuous selection, a stable cell line overexpressing METTL3 is obtained.
   1.6 Fluorescence Recovery after Photobleaching (FRAP) Experiment:

FRAP experiments are conducted using a TCS SP8 confocal microscope (Leica Microsystems). The fluorescence changes in HCC cells transfected with EYFP-METTL3 plasmid, after different treatments (shSMAD3, TGF-β stimulation), are observed in live cells.

### 1.7 Reporter Gene Assay

1) The 1 kilobyte (kb) promoter region of ITIH1 is cloned into the pGL4.17 vector (purchased from Promega), and sequencing is performed to verify the successful construction of the plasmid.
2) This construct is co-transfected with Renilla luciferase-thymidine kinase Promoter vector abbreviated as pRL-TK (purchased from Promega) into HCC cell lines (HLF, 97H). A group transfected with Smad-binding element luciferase reporter vector (SBE-luc) is established as a positive control for signaling pathway activation.
3) The Dual-Glo™ Luciferase Assay System (Promega) is used to measure the luminescence values in each well. Quantitative analysis of the luminescence values is performed to assess the activity of the target gene or regulatory sequences.

### 1.8 RNA Pulldown Experiment

1) Vector construction: Plasmids expressing MS2 RNA hairpin structures (such as pMS2) and plasmids expressing MS2 protein fused with ITIH1 and its 3′-untranslated region (3′-UTR) sequence are constructed.
2) Cell transfection: These plasmids are transfected into HEK-293T cells.
3) Ribonucleoprotein (RNP) complex formation: In the cells, MS2-ITIH1 and 3′-UTR sequence RNP complexes are formed.
4) Affinity purification: Affinity purification is performed using glutathione (GSH) agarose beads to capture the MS2-ITIH1 and 3′-UTR sequence fusion proteins along with their bound RNAs.
5) Protein detection: Western blot analysis is used to detect proteins associated with the MS2 complexes.

### 1.9 Transwell Experiment

1) In the invasion experiment, 40 μL of Matrigel matrix is added.
2) Cells in the logarithmic growth phase and at an appropriate density are harvested using trypsin digestion, resuspended in serum-free medium, counted, and adjusted to a concentration of 2×10⁵ cells/mL.
3) For transwell chambers without Matrigel, 100 μL of the cell suspension (containing 20,000 cells) is slowly added to the upper transwell chamber. For transwell chambers with Matrigel, 200 μL of the cell suspension is slowly added to the upper transwell chamber.
4) Fresh culture medium containing 10% fetal bovine serum is added to the lower transwell chamber, ensuring no air bubbles are introduced, and the chambers are placed in a cell culture incubator for 24 hours.
5) The culture medium is aspirated from both the upper and lower chambers, and cells adhering to the upper surface of the membrane are carefully removed using cotton swabs, repeating this process 2-3 times.
6) In the lower transwell chamber, 600 μL of 4% paraformaldehyde solution is added for fixation at room temperature for 20 min.
7) After removing the paraformaldehyde, 600 μL of 0.4% crystal violet staining solution is added to the lower transwell chamber for staining at room temperature for 10-15 min. Excess crystal violet is washed off with tap water, the membrane is dried with a cotton swab, and images are captured under a microscope by randomly selecting at least two fields.

### 1.10 Scratch Experiment (i.e., Wound Healing Experiment)

1) Cells in the logarithmic growth phase and at an appropriate density are harvested using trypsin digestion and counted.
2) One million cells are seeded into a 6-well plate and evenly distributed by gently shaking the plate to ensure uniform cell growth.
3) On the next day, when cell density reaches approximately 90%, a 10 μL pipette tip is used to scratch a “+” shaped line across each well, ensuring the lines are straight and of uniform width.
4) The medium is replaced with serum-free medium, and the wells can be washed once with PBS. A position for image capture is marked, and images are taken at 0 hours using a microscope and recorded.
5) After 24 hours, the serum-free medium is replaced again, and images are taken at the previously marked positions using a microscope, recording the images at 24 hours.
6) The migration distance of the cells is calculated, and differences in migration distances are statistically analyzed.

### 1.11 Plasmid Extraction

The basic steps are as follows.
1) Bacterial culture is collected by centrifugation at 12,000 rpm for 1 min. The supernatant is discarded to obtain the bacterial pellet (for long-term storage, 500 μL of bacterial culture is mixed with an equal volume of 40% glycerol before centrifugation and stored at −80° C.).
2) 500 μL of K1 solution is added, and the bacterial pellet is resuspended using a pipette.
3) The bacterial suspension is transferred to a new and clean 2 mL EP tube, 500 μL of K2 solution is added, and the mixture is gently inverted 6-10 times and incubated at room temperature for 3 min.
4) Then, 700 μL of K4 solution is added, the mixture is gently inverted 6-8 times, and centrifuged at 12,000 rpm for 5 min.
5) The supernatant is collected, an appropriate amount of isopropanol is added, and the solution is passed through a column.
6) 500 μL of endotoxin removal buffer is added, left at room temperature for 2 min, and centrifuged at 12,000 rpm for 30 s.
7) 600 μL of W2 solution is added, and the mixture is centrifuged at 12,000 rpm for 30 s; this step is repeated once.
8) The mixture is centrifuged at 12,000 rpm for 2 min to remove any liquid from the adsorption membrane.
9) A new 1.5 mL EP tube containing the adsorption column is prepared, 100 μL of Tris-EDTA (TE) buffer is added, and it is left for 5 min before being centrifuged at 12,000 rpm for 2 min.
10) The plasmid concentration is measured and the sample is stored for further use.

### 1.12 Subcutaneous Tumor Formation Experiment

1) Four-week-old male Balb/c-nu nude mice are purchased from Beijing Huafukang Bioscience Co., Inc. and housed in the specific pathogen free (SPF)-grade animal facility at Tongji Hospital affiliated to Tongji Medical College of Huazhong University of Science & Technology. All animal procedures comply with the guidelines set by the Ethics Committee.
2) Cells in good growth condition and at an appropriate density are harvested using trypsin digestion, counted, and adjusted to a concentration of 1×10⁷ cells/mL.
3) After randomly dividing the nude mice into groups, 100 μL of cell suspension is subcutaneously injected into the axilla or dorsal side of the lower limbs using an insulin syringe.
4) The nude mice are observed 2-3 times per week, and once subcutaneous tumors form, their body weight is measured weekly.
5) Every three days, the health status of the nude mice is monitored. Once subcutaneous tumors develop, tumor sizes are measured twice a week using calipers.
6) When the subcutaneous tumors reach an appropriate size, the mice are euthanized, and the tumors are removed and recorded along with relevant data and images. Portions of the tumor tissues are placed in nuclease-free EP tubes and rapidly stored at −80° C. (for RNA and protein extraction), while the remaining tissues are immersed in 4% paraformaldehyde for later use.

### 1.13 Orthotopic Tumor Implantation Experiment

1) Four-week-old male Balb/c-nu nude mice are purchased as mentioned above.
2) Cells in good growth condition and at an appropriate density are harvested using trypsin digestion, counted, and adjusted to a concentration of 1×10⁷ cells/mL.
3) After randomly dividing the nude mice into groups, 100 μL of cell suspension is subcutaneously injected into the axilla or dorsal side of the lower limbs using an insulin syringe. Once subcutaneous tumors reach the size of a soybean, the mice are euthanized under anesthesia, and the subcutaneous tumors are collected.
4) Under sterile conditions, the tumors are cut into 1 cubic millimeter (mm³) pieces. New nude mice are anesthetized, and their abdomens are opened to expose the liver. The small tumor pieces are implanted into the liver using a cannula needle if necessary, ensuring thorough hemostasis before closing the skin with sutures.
6) Six weeks later, the mice are euthanized under anesthesia, and the liver and lungs are collected to document intrahepatic metastasis and lung metastasis (both macroscopic observation and H&E staining).

### 1.14 Hydrodynamic Experiment

1) Four-week-old male C57BL/6J mice are purchased.
2) For establishing the hydrodynamic HCC model, plasmids pT3-EF1α-myr-AKT-HA combined with pT3-EF1α-ΔN90-β-catenin and pT3-EF1α-ITIH1 are used. The total pT3 plasmid is mixed with the sleeping beauty transposon plasmid (pCMV-SB) at a ratio of 25:1.
3) After random grouping, each C57BL/6J mouse receives approximately 2 mL of the mixture containing 20 μg of pT3 plasmids via tail vein injection.
4) Liver samples are collected for analysis about six weeks after the procedure.

### 1.14 Graphing and Statistical Analysis Methods

GraphPad Prism 6 software is used for graphing and statistical analysis, employing Student's t-test or one-way ANOVA. A two-tailed P-value less than 0.05 is considered statistically significant.

### 1.16 Purification of ITIH1 Protein

1) The full-length ITIH1 gene is cloned into the pinfuse purification expression vector.
2) The pinfuse-ITIH1 plasmid is transfected into 293T cells. Twelve hours post-transfection, the medium is replaced with serum-free medium. Twenty-four hours after changing the medium, the supernatant is collected for protein purification.
3) Five to ten volumes of purified water (5-10 mL) are passed through the purification column (Wuhan Huiyan Bio) to wash away the 20% ethanol used for storage.
4) Five to ten volumes of equilibration buffer (5-10 mL) are passed through the column to ensure that the composition and pH of the liquid in the column match those of the sample.
5) After centrifugation filtration (0.45 microns), the sample is applied to the purification column.
6) Five to ten volumes (5-10 mL) of elution buffer are used to elute the protein, collecting the eluates. Following elution, neutralization buffer (100 μL added per 1 mL of eluate) is used to adjust the pH of the eluates to neutrality.
7) After neutralization, the eluates are concentrated using an ultrafiltration column (Millipore, 50 kilodaltons abbreviated as kDa) by centrifugation at 3500 g/min. The column is washed five times with PBS, and finally, residual proteins are recovered from the column using PBS, followed by measuring the purity of the proteins.

### Solution Preparation:

Equilibrium buffer: 0.02 moles per liter (M) PBS, pH: 7.0-7.44, stored at 4° C.
0.2 mol/L Na₂HPO₄.12H₂OH (358.14 g/mol)
0.2 mol/L Na₂HPO₄.2H₂O (156.01 g/mol)
0.2 mol/L PB (PH=7.4-7.5)=81 mL
0.2 mol/L Na₂HPO₄.12H₂O+19 mL 0.2 mol/L Na₂HPO₄.2H₂O

### 2. Results and Analysis

### 2.1. ITIH1 is Down-Regulated by TGF-β Stimulation.

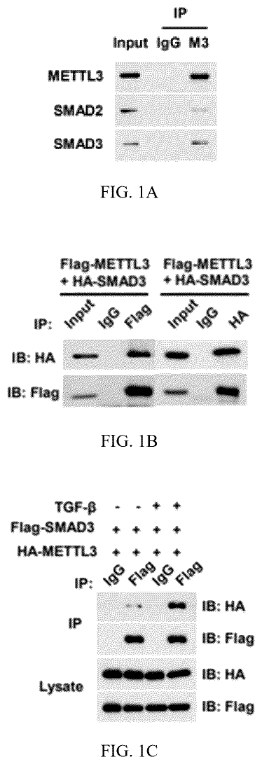

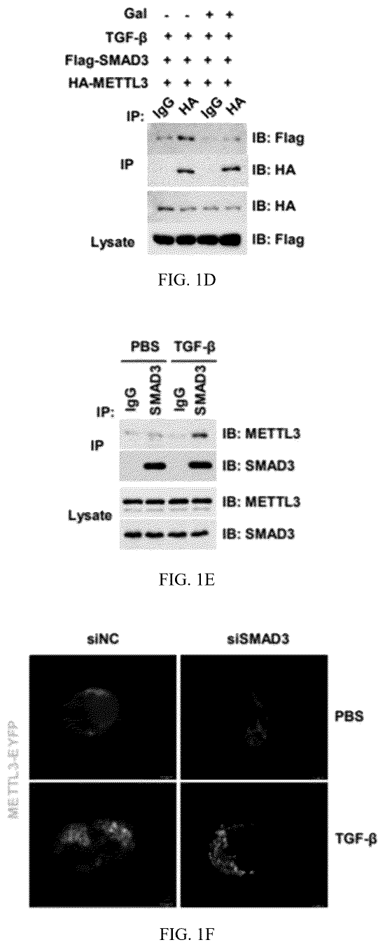

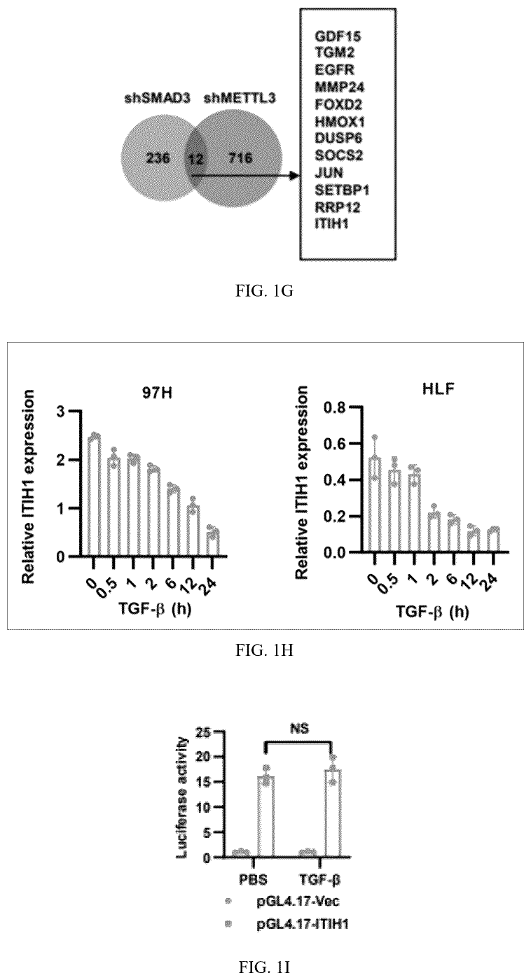

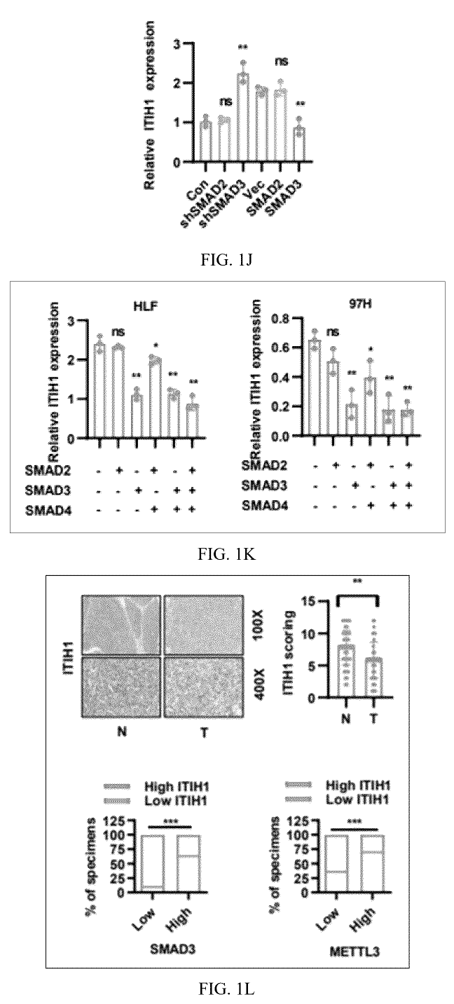

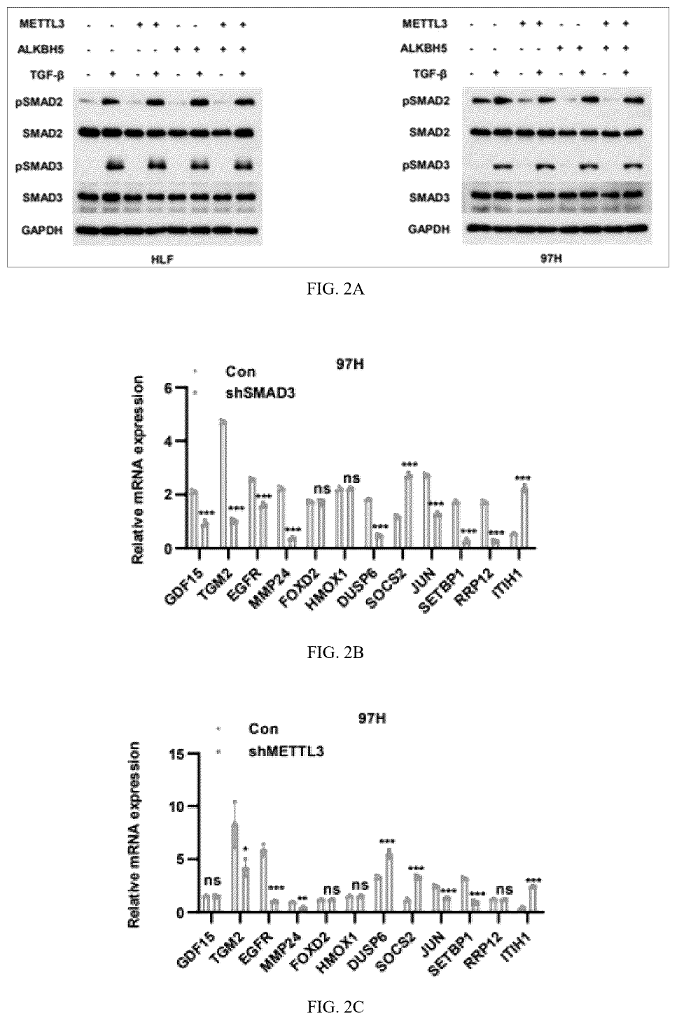

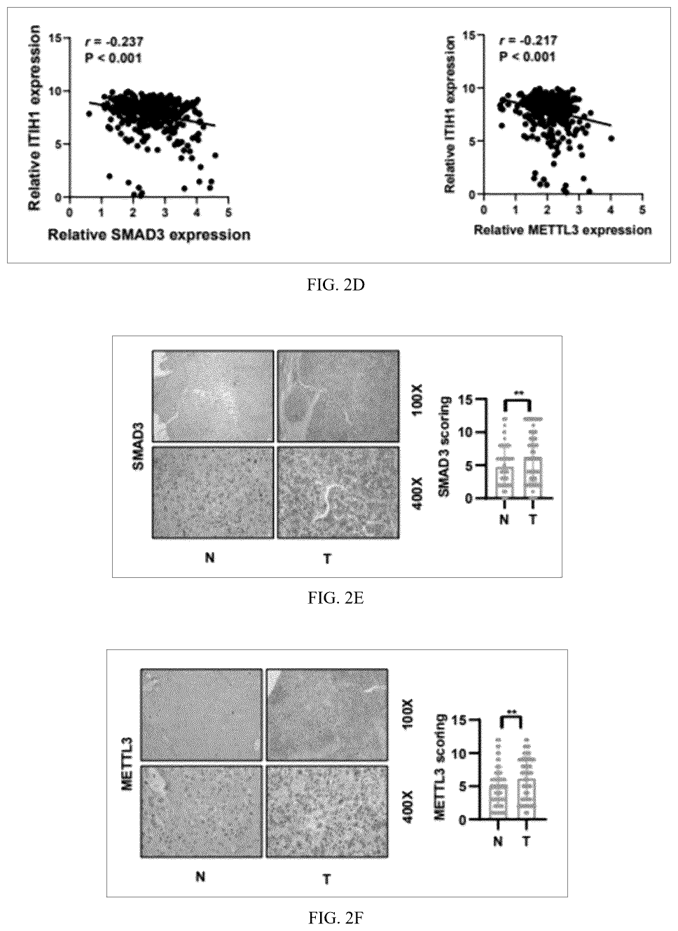

It has been reported in previous studies that the interaction between SMAD2/3 and the m6A “writer” complex promotes m6A deposition on a subset of transcripts. The disclosure subsequently confirms the association between SMAD3 and METTL3 (a key component of the m6A “writer” complex) during HCC progression (FIG. 1A and FIG. 1B). Activation of the TGF-β-induced SMAD complex is further shown to enhance the interaction between SMAD3 and METTL3 (FIG. 1C). In contrast, galunisertib (LY2157299, a selective inhibitor of TGFβR1) treatment leads to a decrease in binding capacity (FIG. 1D). This result is verified by the endogenous IP experiment (FIG. 1E). METTL3 and other members of m6A methyltransferase complex undergone liquid-liquid phase separation (LLPS), and the function of METTL3 condensate is closely related to METTL3. According to the disclosure, it is observed that the knocking down of SMAD3 weakens the formation of METTL3 aggregates, but the stimulation of TGF-β promotes this process (FIG. 1F), which indicates that SMAD3 may promote the formation of METTL3 aggregates, thus enhancing the functional regulation of the “writer” complex. Conversely, the key proteins (METTL3 and ALKBH5) that mediate m6A modification cannot change the activation of TGF-β signaling pathway (FIG. 2A). In order to further explore how SMAD3 and METTL3 regulate the progression of HCC, the disclosure uses RNA sequencing (RNA-seq) data of SMAD3 or METTL3 knockdown in 97H cells (FIG. 1G). These 12 genes co-regulated by SMAD3 and METTL3 are subsequently verified by qRT-PCR in 97H cells (FIG. 2B and FIG. 2C). ITIH1 is the primary target because it is regulated by SMAD3 and METLL3 at the same time. In HLF and 97H cells, a time-dependent decrease in ITIH1 mRNA levels is observed upon TGF-β stimulation (FIG. 1H). As SMAD protein is used as a transcription complex to regulate the expression of the downstream target, the disclosure also uses reporter gene analysis to check whether ITIH1 is regulated by SMAD3 transcription. The results show that TGF-β stimulation does not affect ITIH1 transcription, indicating that the reduction in ITIH1 expression upon SMAD3 knockdown is independent of its transcriptional activity (FIG. 1I). Therefore, TGF-β may regulate ITIH1 at the post-transcriptional level. In order to explore the function of SMAD protein, the effects of overexpressing SMAD2, SMAD3, and SMAD4 are tested, and SMAD3 is demonstrated to be indispensable for the TGF-β-induced downregulation of ITIH1 (FIG. 1J and FIG. 1K). According to predictions from GEPIA, the expression of ITIH1 in HCC samples is negatively correlated with that of SMAD3 and METTL3 (FIG. 2D). IHC staining of three proteins in paraffin-embedded HCC tissues obtained from Tongji Hospital affiliated to Tongji Medical College of Huazhong University of Science & Technology further confirms the negative correlation between ITIH1 and SMAD3/METTL3 (FIG. 1L, FIG. 2E, and FIG. 2F).

These results suggest that TGF-β-SMAD3 may promote the formation of METTL3 aggregates and regulate the level of ITIH1 mRNA.

2.2 METTL3 Regulates the Expression of ITIH1 in a m6A-Dependent Manner.

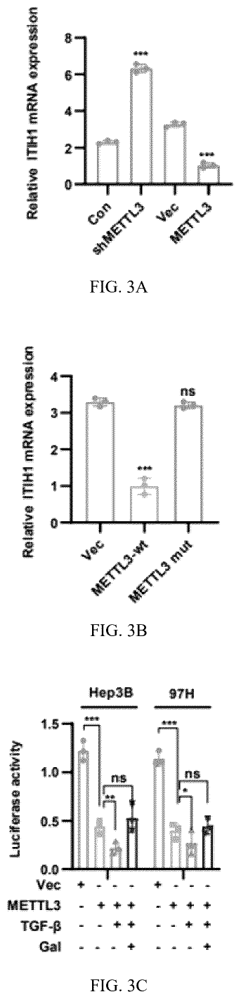

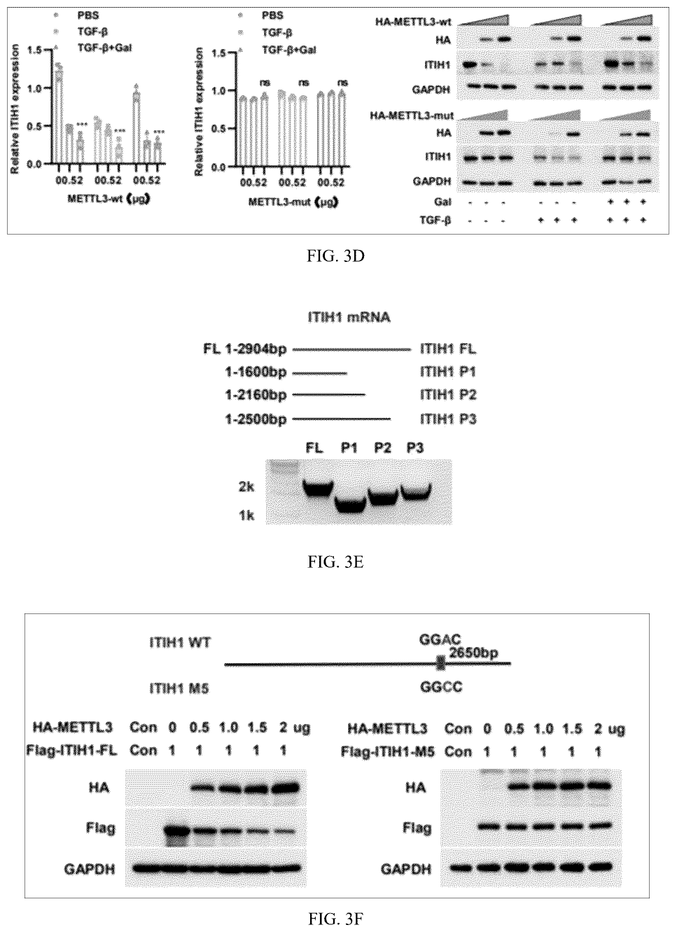

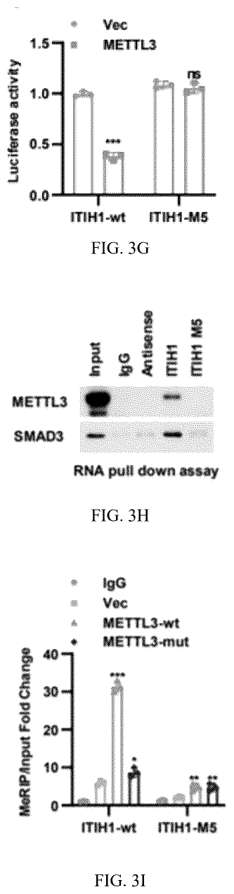

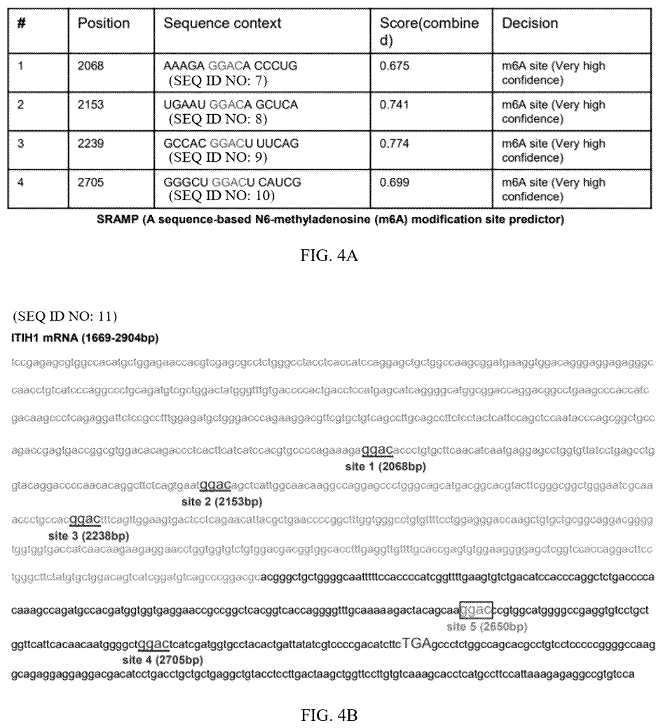

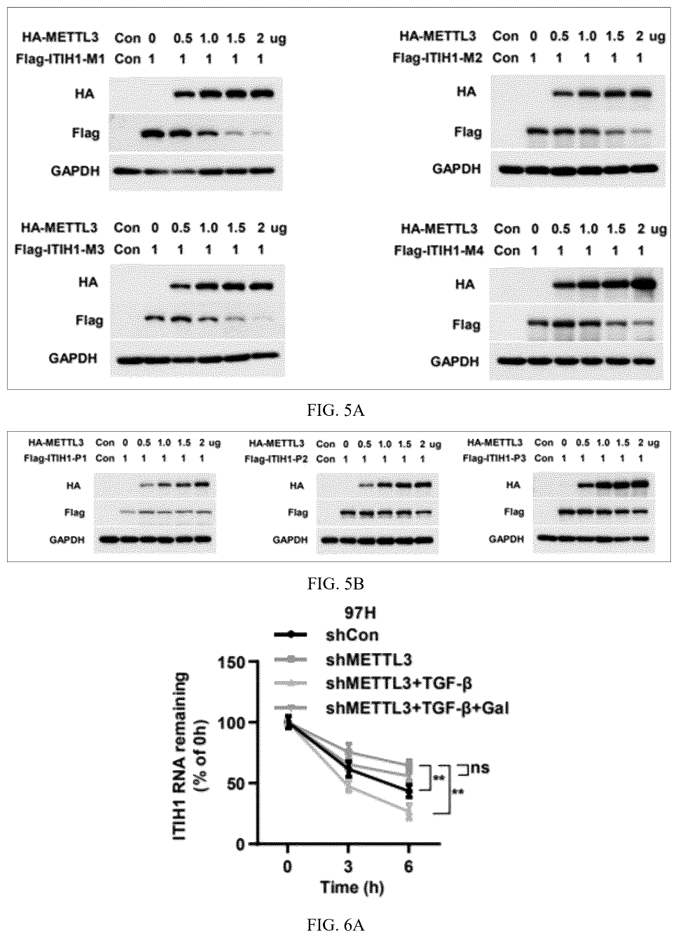

In order to find out how METTL3 regulates the expression of ITIH1, the relative mRNA levels of ITIH1 are examined in 97H cells upon knockdown or overexpression of METTL3. The results show that the expression of ITIH1 is negatively regulated by METTL3 (FIG. 3A). Since METTL3 is a key component of the m6A “writer” complex, an inactive METTL3 mutant (METTL3-mut) is used to test whether METTL3-mediated regulation of ITIH1 is dependent on m6A methylation. When METTL3-mut is expressed, no significant change in ITIH1 mRNA levels is observed (FIG. 3B). Subsequently, a psiCHECK2 plasmid containing the ITIH1 sequence is constructed. Dual-luciferase reporter assays indicate that METTL3 may bind to ITIH1 mRNA. This interaction is enhanced by TGF-β stimulation and reduced by galunisertib treatment (FIG. 3C). These data indicate that METTL3 may regulate ITIH1 in a m6A-dependent manner. In addition, ITIH1 expression is downregulated by METTL3 in a dose-dependent manner, and this effect is enhanced by TGF-β stimulation. However, when the catalytically inactive METTL3-mut is expressed, TGF-β stimulation no longer affects ITIH1 expression (FIG. 3D). According to SRAMP analysis, four predicted m6A sites (with very high confidence) are identified within the ITIH1 mRNA. To confirm whether these sites are responsible for METTL3-mediated regulation, point mutations are introduced at the putative m6A sites, generating ITIH1-M1, ITIH1-M2, ITIH1-M3, and ITIH1-M4 mutants. When co-transfected with increasing amounts of METTL3, no significant change in the levels of these ITIH1 mutants is detected (FIG. 5A). Next, truncated ITIH1 mutants are used to confirm that the m6A modification site is located between 2500 bp and 2904 bp (FIG. 3E and FIG. 5B). However, by using the same strategy, another m6A site (with high confidence) is identified at 2650 bp (ITIH1-M5) (FIG. 3F, FIG. 3G, and FIG. 4B). RNA pulldown experiment further confirms that neither METTL3 nor SMAD3 binds to the ITIH1-M5 mRNA (FIG. 3H). Me-RIP experiments show that the m6A modification in ITIH1-M5 mediated by METTL3 is much lower than that in ITIH1-wt (FIG. 3I). Collectively, these data reveal that METTL3 catalyzes m6A modification on ITIH1 mRNA, and this modification is functionally linked to the regulation of ITIH1 expression.

2.3 YTHDF2 Promotes the Degradation of ITIH1 mRNA in a m6A-Dependent Manner.

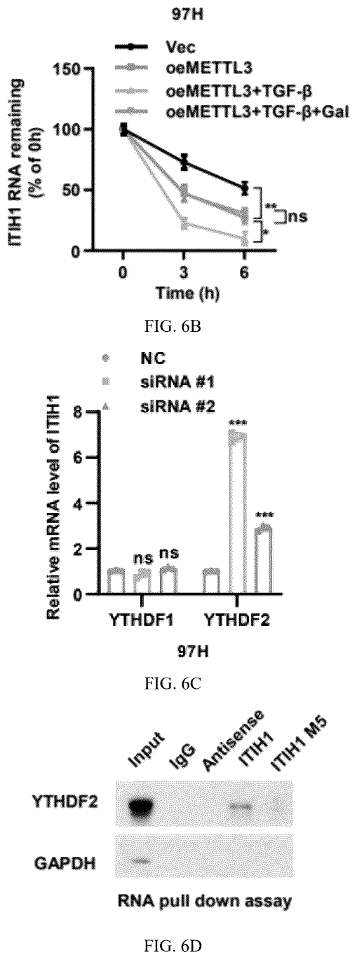

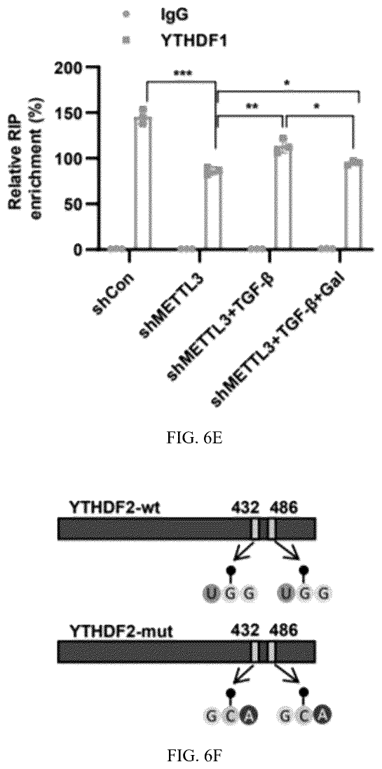

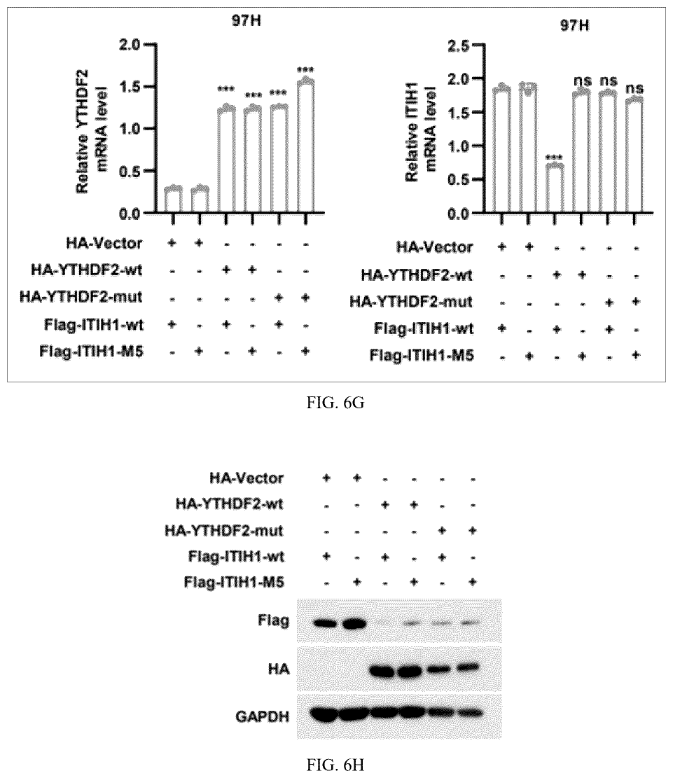

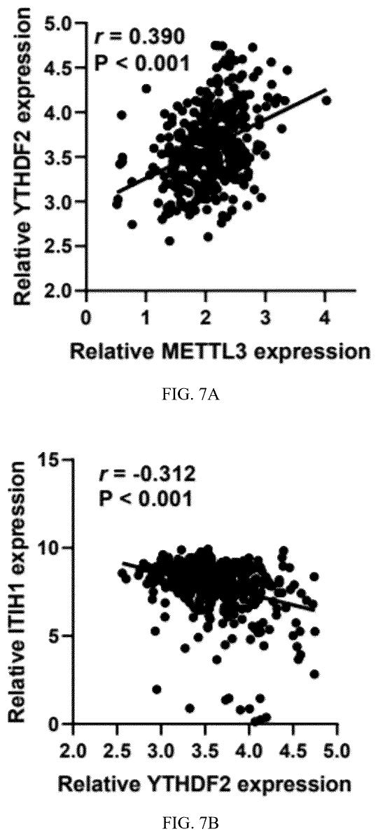

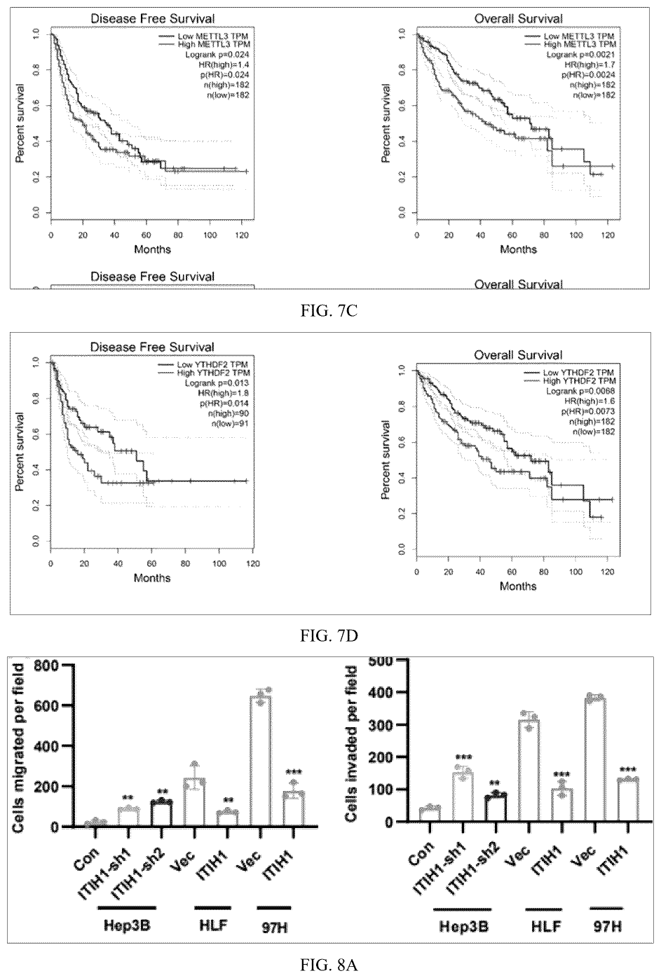

Previous studies have shown that m6A modification has been proved to regulate the fate of mRNA. It is investigated here how m6A modification affects ITIH1 mRNA. After transcription is blocked by actinomycin D, the degradation rate of ITIH1 mRNA is reduced when METTL3 is knocked down, whereas it is increased upon METTL3 overexpression. However, TGF-β stimulation is observed to enhance ITIH1 degradation, while treatment with galunisertib is found to counteract this effect (FIG. 6A and FIG. 6B), indicating that METTL3 regulates the degradation of ITIH1 mRNA. It has been reported that YTHDF2 recruits the mRNA decay machinery to reduce mRNA stability. When YTHDF2 is knocked down, ITIH1 mRNA levels are increased. In contrast, the other m6A “reader” YTHDF1 is shown to have minimal impact on ITIH1 mRNA (FIG. 6C). RNA pulldown experiment shows that YTHDF2 binds to ITIH1-wt, but not to ITIH1-M5 mRNA (FIG. 6D). In order to further confirm the association between YTHDF2 and ITIH1 mRNA, the RIP experiment is carried out, and a reduction in YTHDF2 enrichment is observed upon METTL3 knockdown. TGF-β stimulation is shown to promote YTHDF2 binding, whereas this effect is abolished by galunisertib treatment (FIG. 6E). Next, the disclosure uses YTHDF2-mut (W432A and W486A), which no longer recognizes the m6A modification (FIG. 6F). Western blot and qPCR analyses reveal that YTHDF2-wt, but not YTHDF2-mut, negatively regulates the expression of ITIH1-wt, while YTHDF2-wt has little effect on the expression of ITIH1-M5 (FIG. 6G and FIG. 6H). GEPIA analysis indicates that YTHDF2 expression is negatively correlated with ITIH1 expression and positively correlated with METTL3 expression (FIG. 7A and FIG. 7B). In addition, DFS and OS of METTL3 and YTHDF2 show a similar trend (FIG. 7C and FIG. 7D).

These results demonstrate that YTHDF2 recognizes the METTL3-mediated m6A modification on ITIH1 mRNA and regulates its stability.

### 2.4 ITIH1 Inhibits the Development of HCC In Vitro and In Vivo.

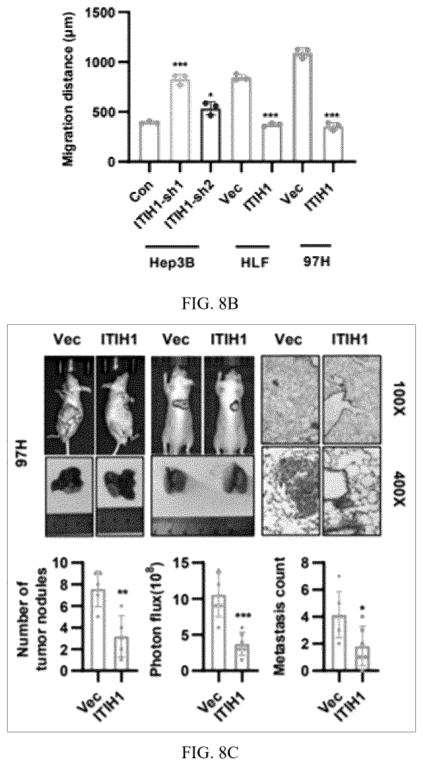

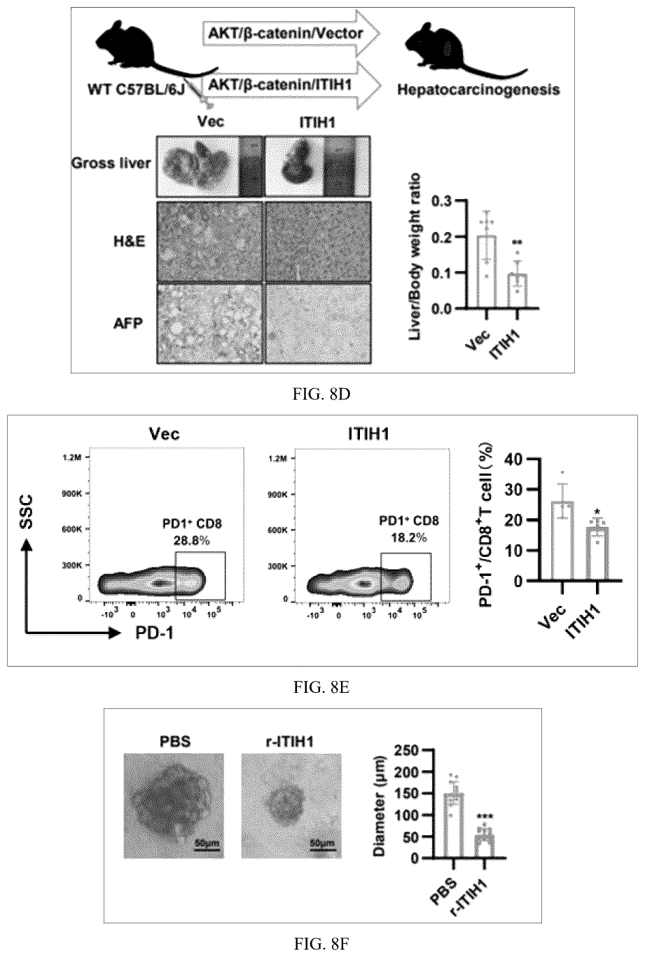

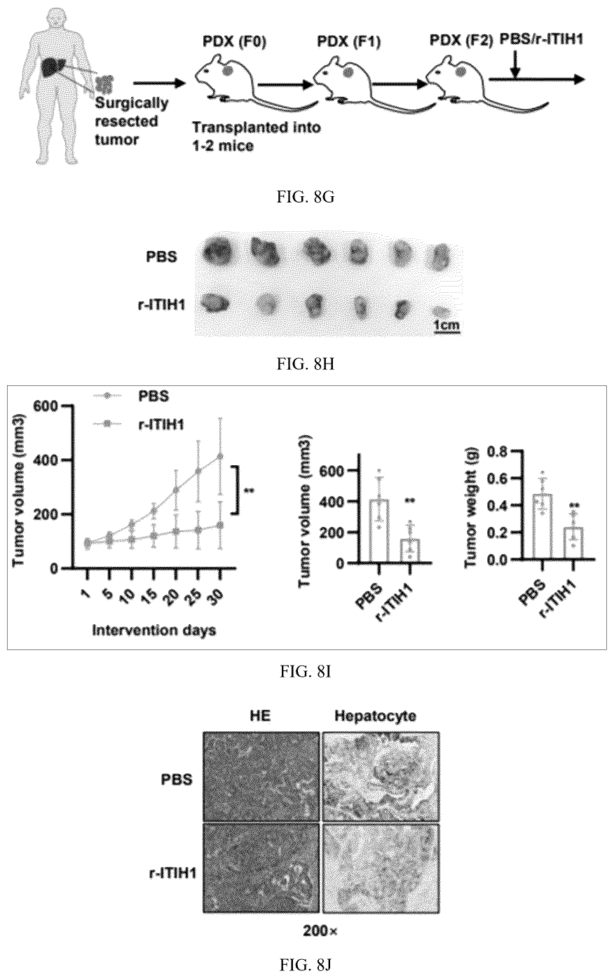

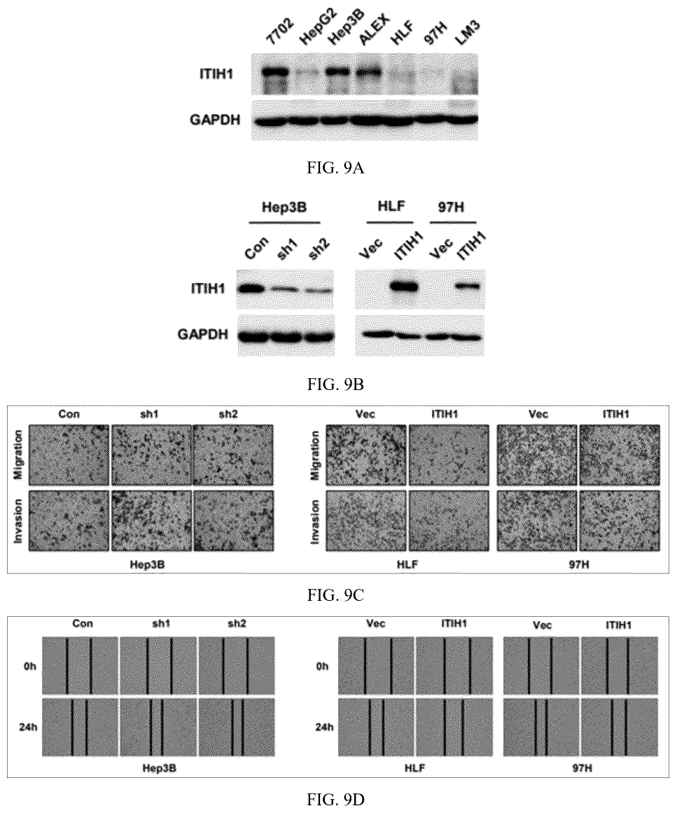

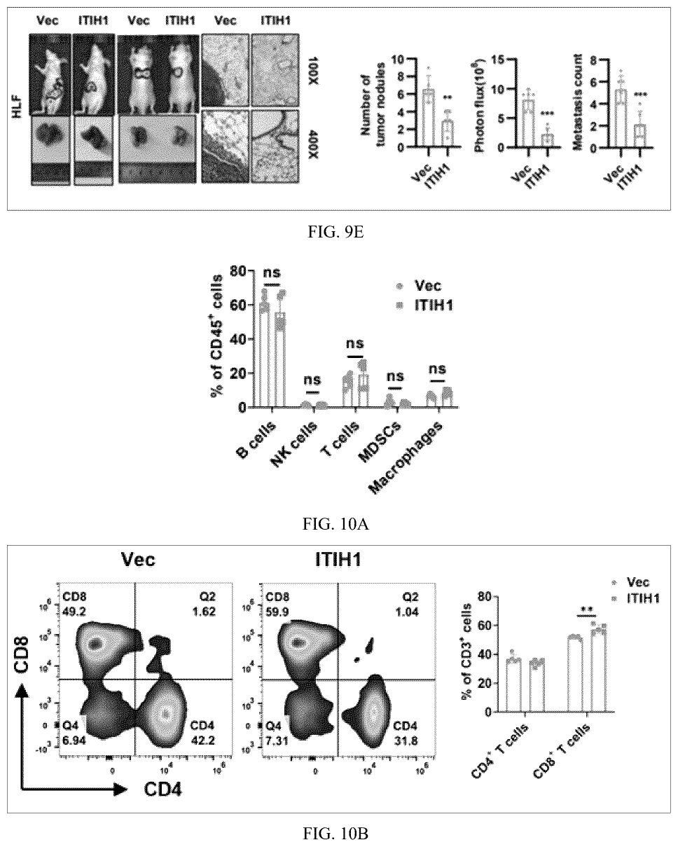

In order to explore the role of ITIH1 in HCC, its expression levels are examined across different cell lines (FIG. 9A). In Hep3B cells expressing high levels of ITIH1, shRNA-mediated knockdown of ITIH1 is shown to increase cell migration and invasion. In contrast, overexpression of ITIH1 in HLF and 97H cells is found to suppress cell motility (FIG. 8A, FIG. 9B, and FIG. 9C). Similar effects are also observed in wound healing experiments (FIG. 8B and FIG. 9D). In order to evaluate the tumor inhibitory effect of ITIH1 in HCC in vivo, different xenograft models are employed in the disclosure. First, an orthotopic model is used to assess intrahepatic metastasis five weeks after cell inoculation. Injection of cells overexpressing ITIH1 is found to significantly reduce the ability of HCC cells to form secondary lesions in the liver (FIG. 8C and FIG. 9E). In a lung metastasis model, control or ITIH1-overexpressing cells are injected into mice via the tail vein. Six weeks after injection, fewer metastatic lesions are observed in mice injected with ITIH1-overexpressing cells (FIG. 8C and FIG. 9E). In order to evaluate the influence of ITIH1 on the development of HCC, HTVi is performed using AKT/β-catenin plasmids. Mice are observed to develop HCC 16 weeks after injection. IHC staining reveals that AFP expression is reduced in the ITIH1-overexpressing group, and the liver-to-body weight ratio is lower in this group compared to the control (FIG. 8D). In addition, tumor-infiltrating immune cells are analyzed in this model. No significant differences are observed between the two groups in the populations of macrophages, myeloid-derived suppressor cells (MDSCs), B cells, NK cells, and T cells within the tumor microenvironment (FIG. 10A). Considering the key role of T cells in tumor killing, flow cytometry analysis is conducted on T cell subsets, and a higher proportion of CD8⁺ T cells is found within the tumor-infiltrating T cell population in the ITIH1-overexpressing group (FIG. 10B). Additionally, a reduced proportion of PD-1⁺CD8⁺ T cells is observed after ITIH1 overexpression, suggesting decreased CD8 T cell exhaustion (FIG. 8E). These results indicate that ITIH1 inhibits the development of HCC. Since ITIH1 is a secreted protein located in extracellular matrix, purified r-ITIH1 is subsequently used to evaluate its tumor-suppressive effects in vivo (FIG. 10C). In order to study whether r-ITIH1 protein has therapeutic effect against HCC, the disclosure establishes an organ-like model (also referred to as organoid model) from HCC patients. The r-ITIH1 is shown to significantly inhibit the growth of HCC-like organs (also referred to as HCC organoids) (FIG. 8F). In another HCC patient-derived xenograft (PDX) model (FIG. 8G), r-ITIH1 is administered via peritumoral injection. Notably, r-ITIH1 is found to significantly suppress the growth of PDX tumors (FIG. 8H-FIG. 8J).

In summary, these data demonstrate that ITIH1 functions as a tumor suppressor during HCC progression.

### 2.5. Interaction Between ITIH1 and Integrin α5β1

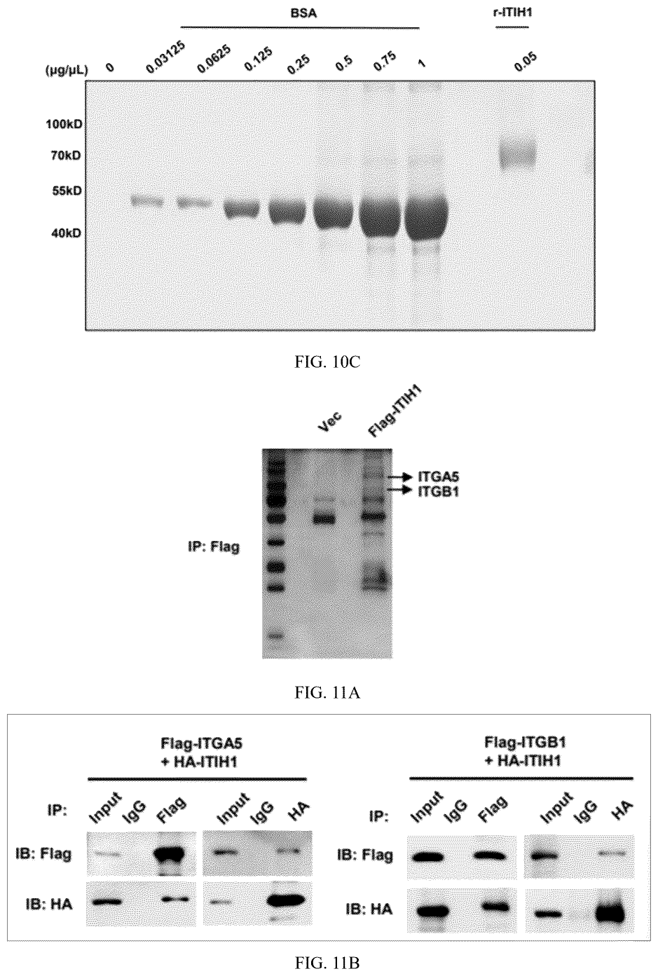

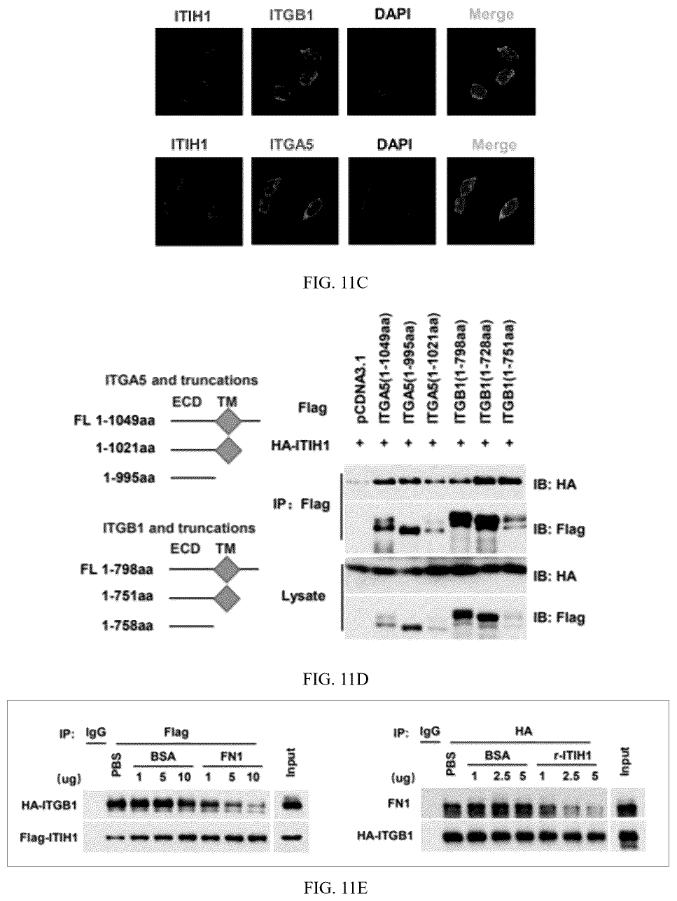

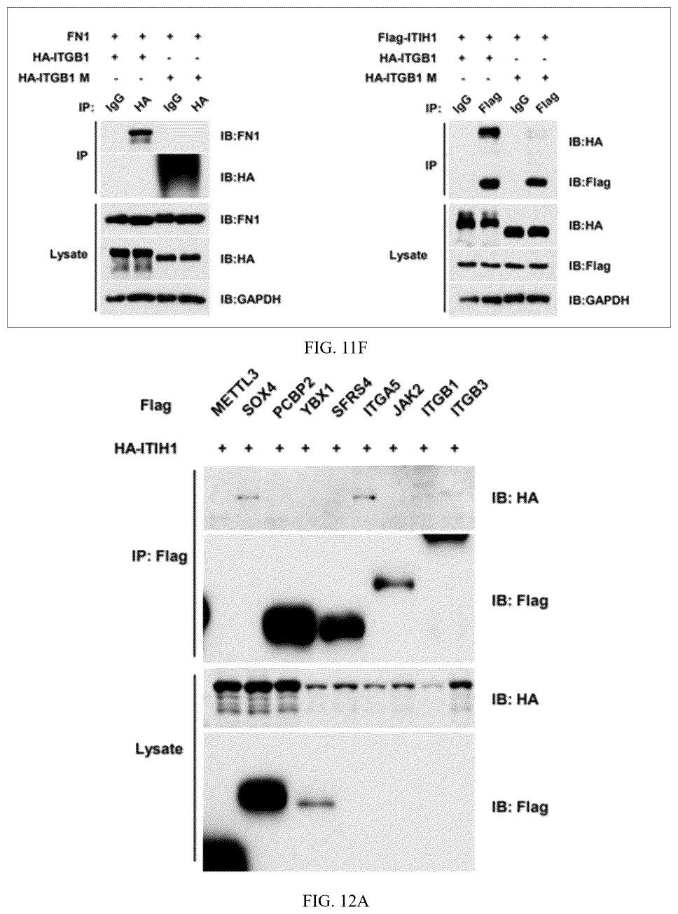

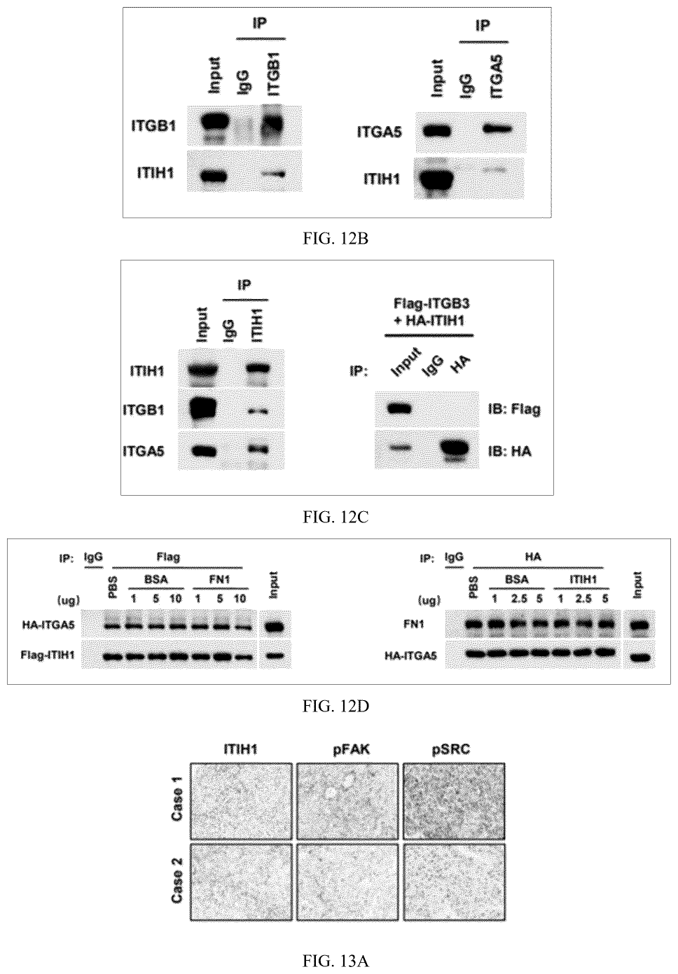

The potential mechanism underlying ITIH1-induced suppression of HCC metastasis and progression is subsequently explored. Potential ITIH1-interacting partners are identified using IP coupled with mass spectrometry (MS) (FIG. 11A). Integrin family proteins are validated and analyzed (FIG. 12A). As a functional complex, integrins are composed of a and β subunits. ITGB1 and ITGA5 are paired subunits that transmit extracellular signals to cells. Co-IP results show that ITIH1 exhibits higher binding affinity to ITGB1 and ITGA5 than to other integrin family proteins (FIG. 11B and FIG. 12B). IF staining shows that ITIH1 is colocalized with ITGB1 and ITGA5 on the cell membrane (FIG. 11C). Further analysis indicates that ITIH1 interacts with the extracellular domains of ITGB1 and ITGA5 (FIG. 11D). It has been reported that fibronectin (FN) is an important ligand of ITGB1/ITGA5 complex, and its binding leads to activation of the integrin/FAK signaling pathway. Subsequently, the disclosure confirms that ITIH1 binds to ITGB1 in a dose-dependent and competitive manner, while no effect of ITIH1 on the ITGA5/FN complex is observed (FIG. 12C). In order to further verify this observation, an ITGB1 mutant (ITGB1 M) is generated, in which amino acids 130-240 are deleted. This mutant is impaired in FN binding but remains localized on the cell surface. The ITGB1 mutant is found to interact with neither FN nor ITIH1, indicating that both FN and ITIH1 bind to the same region of ITGB1 (FIG. 11F).

Collectively, these findings demonstrate that ITIH1 functions as a novel ligand for ITGB1/ITGA5 and competes with FN for binding to ITGB1.

2.6. Expression of ITIH1 is Negatively Correlated with FAK Activation in HCC Patients.

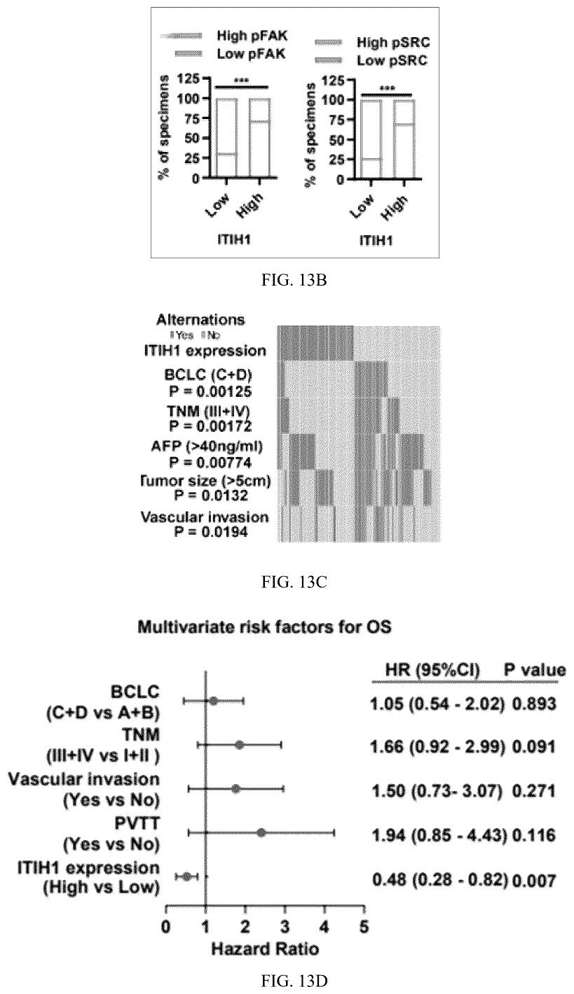

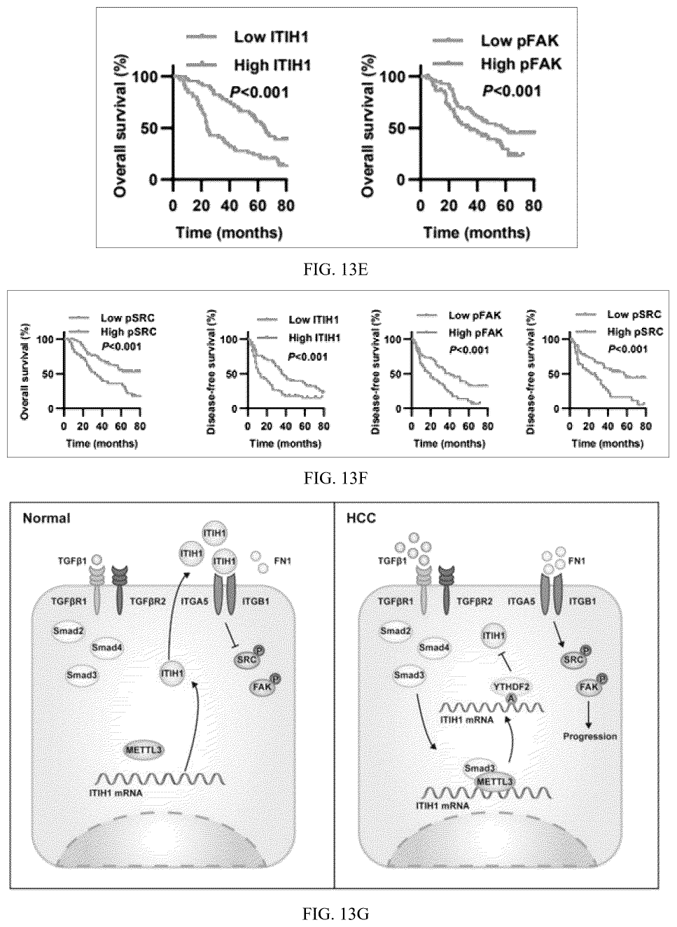

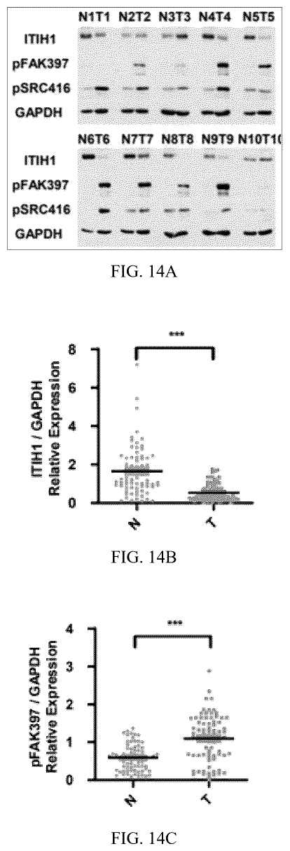

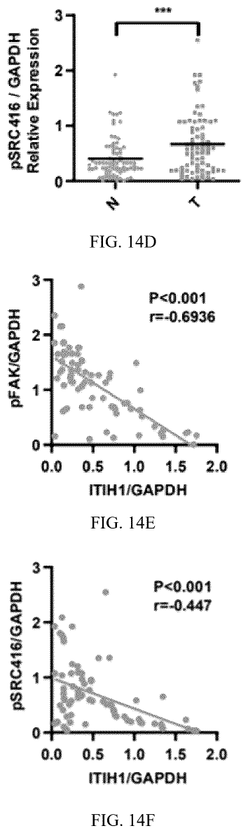

IHC analysis shows that the expression of ITIH1 is negatively correlated with the activation of FAK and SRC in HCC samples (FIG. 13A and IG. 13B). To further confirm this correlation, the levels of relevant proteins are examined in tumor tissues and adjacent non-tumor tissues by WB analysis (FIG. 14A). ITIH1 protein levels are found to be decreased in tumor tissues, whereas pFAK (Tyr397) and pSRC (Tyr416) levels are increased in tumor tissues (FIG. 14B-FIG. 14D). Overall, WB analysis indicates that ITIH1 protein levels are negatively correlated with pFAK and pSRC levels in HCC tissues (FIG. 14E and FIG. 14F). When these findings are combined with clinical data from HCC patients, low ITIH1 expression is observed to be associated with advanced BCLC stage (P=0.00125), advanced TNM stage (P=0.00172), high AFP expression (P=0.00774), larger tumor size (P=0.0132), and increased vascular invasion (P=0.0194, FIG. 13C). Univariate regression analysis of clinicopathological features, including ITIH1 expression, shows that BCLC stage, TNM stage, vascular invasion, portal vein tumor thrombus (PVTT), and ITIH1 expression are associated with OS. After multivariate regression analysis of these factors, ITIH1 expression is identified as an independent predictor of OS (FIG. 13D). Consistent with this finding, high ITIH1 protein levels, low pFAK (Tyr397) levels, and low pSRC (Tyr416) levels are associated with better DFS and OS (FIG. 13E and FIG. 13F). In summary, the study of the disclosure reveals that ITIH1, whose expression is coordinately regulated by TGF-β stimulation and m6A modification, inhibits HCC metastasis by suppressing the integrin/FAK signaling pathway, and the efficacy of r-ITIH1 in inhibiting HCC metastasis is preliminarily validated (FIG. 13G).

In the process of cancer development, TGF-β signaling pathway plays an important role in different stages of cancer. M6A mRNA modification also affects tumor progression through various mechanisms including LLPS. SMAD2/3 promotes the binding of m6A methyltransferase complex with some transcripts involved in early cell fate determination. However, the relationship between TGF-β signaling and m6A modification in HCC progression remains poorly understood. First, the interaction between these pathways is confirmed in HCC cells. TGF-β stimulation is observed to promote the LLPS of METTL3, and SMAD3 is found to be indispensable for this process. Subsequently, the role of an extracellular matrix (ECM) protein, ITIH1, is validated. ITIH1 is regulated by TGF-β stimulation, but this regulation is shown to be independent of the transcriptional activity of TGF-β. In this study, ITIH1 is identified as a direct downstream target of both TGF-β signaling and m6A modification. Consistent with this finding, ITIH1 downregulation is not induced by TGF-β stimulation when METTL3-mut, rather than METTL3-wt, is expressed. Next, upon translocation of m6A-modified ITIH1 mRNA into the cytoplasm, it is recognized by the reader protein YTHDF2 and subsequently degraded. This mechanism may explain why lower ITIH1 expression is detected in HCC tumor tissues. The specific mechanism by which TGF-β promotes METTL3 LLPS through SMAD3 remains to be further elucidated.

In the process of cancer progression, ECM proteins are generally reported to exert tumor-promoting effects. However, tumor-suppressive ECM components are less explored. In this study, it is found that when ITIH1 is secreted from cells, it is able to compete with FN for binding to the ITGB1/ITGA5 complex, thereby inhibiting the activation of integrin/FAK signaling. Consistent with the observation that high ITIH1 expression is associated with favorable clinical outcomes in HCC patients, ITIH1 overexpression and treatment with r-ITIH1 are shown to suppress tumor metastasis and progression in mouse models without causing significant toxicity, suggesting the potential therapeutic value of ITIH1.

Integrin is a key surface receptor that links intracellular structures with ECM components and transmits critical signals. In tumor cells, paired integrins are known to interact with FN and other ECM proteins to promote cancer progression. In this study, the binding between ITIH1 and the extracellular domains of ITGB1 and ITGA5 is confirmed. Integrins function as heterodimeric complexes composed of α and β subunits. Contrary to the supposed association, ITIH1, as a ligand for the ITGB1/ITGA5 complex, is found to compete with FN for binding to ITGB1, but not to ITGA5. The underlying mechanism involving these three molecules requires further investigation.

According to National Center for Biotechnology Information (NCBI) RNA-seq data, ITIH1 is identified as a protein expressed specifically in the liver. In addition to its therapeutic potential, ITIH1 may also serve as a diagnostic biomarker for HCC.

It should be noted that in the disclosure, the terms “comprising”, “including” or any other variation thereof are intended to cover non-exclusive inclusion, so that a process, method, article or device including a series of elements includes not only those elements, but also other elements not explicitly listed, or elements inherent to such process, method, article or device. Without further limitations, an element defined by the phrase “comprising a . . . ” does not exclude the presence of additional identical elements in a process, method, article, or apparatus that includes the stated element.

The embodiment numbers of the disclosure provided above are merely for descriptive purposes and do not indicate the superiority or inferiority of the embodiments.

The above embodiments are only used to illustrate the technical solutions of the disclosure and not for limiting them. Although the disclosure has been described in detail with reference to the illustrated embodiments, those skilled in the art should understand that the technical solutions of the disclosure can be modified or replaced by equivalents without departing from the purpose and scope of the technical solutions of the disclosure, which should be included in the scope of the claims of the disclosure.

What is claimed is:

1. An application method of inter-alpha-trypsin inhibitor heavy chain 1 (ITIH1), comprising:
preparing a drug by using the ITIH1 for treating hepatocellular carcinoma.

2. The application method as claimed in claim 1, wherein the drug comprises the ITIH1 or a reagent for increasing an expression of the ITIH1.

3. The application method as claimed in claim 2, wherein the reagent comprises a small molecule compound, an antibody drug, a protein, a nucleic acid molecule, a polypeptide, a lipid, a carbohydrate or a combination thereof.

4. The application method as claimed in claim 1, wherein the ITIH1 is used to influence metastasis of hepatocellular carcinoma by competitively binding to an integrin subunit beta 1 (ITGB1) and integrin subunit alpha 5 (ITGA5) complex and inhibiting integrin and focal adhesion kinase (FAK) signaling pathway.

5. The application method as claimed in claim 1, wherein an expression of the ITIH1 is regulated by transforming growth factor-beta (TGF-β) stimulation and N6-methyladenosine (m6A) modification.

6. An application method of a substance taking one of an ITIH1 protein and an ITIH1 gene as a target, comprising:
preparing a drug for treating hepatocellular carcinoma by using the substance; wherein the target is one of an up-regulation target and an activation target, and the drug is indicated for treating hepatocellular carcinoma.

7. A drug, comprising a substance taking one of an ITIH1 protein and an ITIH1 gene as a target, wherein the target is one of an up-regulation target and an activation target, and the drug is indicated for treating hepatocellular carcinoma.
